SEQUENCE LISTING (14 sequences)

SEQ ID NO: 1 - DNA, 23 bp, synthetic construct
  misc_feature 1..23: note = INS_Forward
  source 1..23: mol_type = other DNA
ctgcatcagaagaggccatcaag

SEQ ID NO: 2 - DNA, 21 bp, synthetic construct
  misc_feature 1..21: note = INS_Reverse
  source 1..21: mol_type = other DNA
gggtgtgtagaagaagcctcg

SEQ ID NO: 3 - DNA, 17 bp, synthetic construct
  misc_feature 1..17: note = MAFA_Forward
  source 1..17: mol_type = other DNA
cgcacgctcaagaaccg

SEQ ID NO: 4 - DNA, 25 bp, synthetic construct
  misc_feature 1..25: note = MAFA_Reverse
  source 1..25: mol_type = other DNA
gccagcttctcgtatttctccttgt

SEQ ID NO: 5 - DNA, 24 bp, synthetic construct
  misc_feature 1..24: note = PDX-1_Forward
  source 1..24: mol_type = other DNA
ttcacgagccagtatgaccttcac

SEQ ID NO: 6 - DNA, 22 bp, synthetic construct
  misc_feature 1..22: note = PDX-1_Reverse
  source 1..22: mol_type = other DNA
gaagacagacctgggatgcaca

SEQ ID NO: 7 - DNA, 20 bp, synthetic construct
  misc_feature 1..20: note = NEUROG3_Forward
  source 1..20: mol_type = other DNA
ctaagagcgagttggcactg

SEQ ID NO: 8 - DNA, 20 bp, synthetic construct
  misc_feature 1..20: note = NEUROG3_Reverse
  source 1..20: mol_type = other DNA
ccgagttgaggttgtgcatt

SEQ ID NO: 9 - DNA, 20 bp, synthetic construct
  misc_feature 1..20: note = GAPDH_Forward
  source 1..20: mol_type = other DNA
ctgcaccaccaactgcttag

SEQ ID NO: 10 - DNA, 20 bp, synthetic construct
  misc_feature 1..20: note = GAPDH_Reverse
  source 1..20: mol_type = other DNA
aggcagggatgatgttctgg

SEQ ID NO: 11 - DNA, 20 bp, synthetic construct
  misc_feature 1..20: note = NEUROD1_Forward
  source 1..20: mol_type = other DNA
taagacgcagaagctgtcca

SEQ ID NO: 12 - DNA, 20 bp, synthetic construct
  misc_feature 1..20: note = NEUROD1_Reverse
  source 1..20: mol_type = other DNA
gtccgaggattgagttgcag

SEQ ID NO: 13 - DNA, 17 bp, synthetic construct
  misc_feature 1..17: note = NKX6.1_Forward
  source 1..17: mol_type = other DNA
gaaccgccggaccaagt

SEQ ID NO: 14 - DNA, 21 bp, synthetic construct
  misc_feature 1..21: note = NKX6.1_Reverse
  source 1..21: mol_type = other DNA
gtcgtccgagttgggatccag

(19) United States
(12) Patent Application Publication
(10) Pub. No.: US 2023/0088644 A1
(43) Pub. Date: Mar. 23, 2023

(54) COMPOSITION FOR INDUCING DIFFERENTIATION INTO INSULIN-PRODUCING CELLS, AND USE THEREOF

(71) Applicants: SEOUL NATIONAL UNIVERSITY R&DB FOUNDATION, Seoul (KR); SEOUL NATIONAL UNIVERSITY HOSPITAL, Seoul (KR)

(72) Inventors: Kyong Soo PARK, Seoul (KR); Seung-Ah LEE, Gyeonggi-do (KR); Yong Deok LEE, Seoul (KR); Hakmo LEE, Seoul (KR); Sung Soo CHUNG, Seoul (KR)

(21) Appl. No.: 17/811,403

(22) Filed: Jul. 8, 2022

Related U.S. Application Data
(63) Continuation-in-part of application No. PCT/KR2021/000276, filed on Jan. 8, 2021.

(30) Foreign Application Priority Data
Jan. 9, 2020 (KR) 10-2020-0003309

(51) Int. Cl. C12N 5/071; A61P 3/10

(52) U.S. Cl. CPC C12N 5/0678; A61P 3/10; C12N 2501/40

(57) ABSTRACT

The present invention relates to a composition for inducing differentiation into insulin-producing cells, and a method for inducing differentiation into insulin-producing cells. By using a differentiation inducing composition according to an exemplary embodiment or a differentiation inducing method according to an exemplary embodiment, insulin-producing cells can be prepared in a short period by effectively inducing the differentiation of various types of stem cells into insulin-producing cells, and can be mass-produced in a relatively simple manner, and thus a pharmaceutical composition for preventing or treating diabetes mellitus, comprising insulin-producing cells and/or insulin produced thereby, can be provided.

### TECHNICAL FIELD

The present invention relates to a composition for inducing differentiation into insulin-producing cells, and a method for inducing differentiation into insulin-producing cells.

### BACKGROUND ART

Diabetes mellitus is a chronic metabolic disease characterized by hyperglycemia, and the prevalence of diabetes mellitus is rapidly increasing in both developed and developing countries as a disease associated with metabolic disorders. Diabetes mellitus is broadly divided into two major forms (type 1 and type 2). Type 1 diabetes mellitus is caused by autoimmune destruction of pancreatic beta cells, which usually leads to absolute insulin deficiency. Type 2 diabetes mellitus is caused by interaction of insulin resistance and relative insulin deficiency.

Management of type 2 diabetes mellitus includes lifestyle modification and pharmacological therapy. Pharmacological management of type 2 diabetes mellitus has evolved very rapidly with recent development of new pharmacological agents such as incretin mimetics and SGLT2 inhibitors. Most patients with type 1 diabetes showed absolute insulin deficiency and should be treated with multiple subcutaneous insulin injections of prandial and basal insulin or continuous subcutaneous insulin infusion to mimic physiological insulin response. Pancreas or islet transplantation can normalize glucose levels in type 1 diabetes mellitus but it requires lifelong immunosuppression for graft survival. Another limitation for pancreas or islet transplantation is a limited supply. There have been efforts to differentiate stem cells into insulin-producing cells using embryonic stem cells or induced pluripotent stem cells (iPSC) and attempts to transplant these insulin-producing cells with limited success. Differentiation of embryonic stem cells or iPSC requires several weeks of expensive, multi-stage differentiation protocol and still has safety concern.

Therefore, much research is needed for developing an effective and safe method with a right composition for a short period of time to efficiently induce differentiation of various stem cells into insulin-producing cells.

### DISCLOSURE

### Technical Problem

Thus, the inventors of the present application confirmed that a composition comprising putrescine, glucosamine, and/or nicotinamide efficiently induces differentiation from stem cells into insulin-producing cells within a short period of time, and confirmed an insulin secretory effect in cells in which differentiation was induced, thereby completing the present invention.

An object of the present invention is to provide a composition for inducing differentiation into insulin-producing cells, comprising putrescine alone; or one or more selected from the group consisting of putrescine, glucosamine, and nicotinamide.

Another object of the present invention is to provide a culture medium composition for inducing differentiation into insulin-producing cells, comprising the composition for inducing differentiation and a culture medium.

Still another object of the present invention is to provide a method for inducing differentiation into insulin-producing cells, the method comprising culturing cells in the culture medium composition for inducing differentiation.

Yet another object of the present invention is to provide a pharmaceutical composition for preventing or treating diabetes mellitus in a patient, comprising the composition for inducing differentiation and a mixture of patient-derived blood.

Yet another object of the present invention is to provide a pharmaceutical composition for preventing or treating diabetes mellitus, comprising insulin-producing cells differentiated by the differentiation inducing method.

### Technical Solution

As used herein, “insulin-producing cells” refers to cells capable of producing and secreting insulin and cells capable of performing the same function as pancreatic beta cells, and may be, for example, pancreatic beta cells.

As used herein, “differentiation” means that cells develop into specific cells, and specifically, refers to a phenomenon in which a structure or function is specialized while cells divide, proliferate, and grow, and means that the morphology of function of cells, tissues, and the like of an organism changes in order to carry out the tasks given to them.

As used herein, “stem cells” are cells capable of differentiating into various cells that make up a biological tissue, and the term refers to undifferentiated cells capable of being regenerated to form specialized cells of tissues and organs. The stem cells may be autologous or allogeneic stem cells, and may be derived from a human, a non-human primate, and/or a mammal such as a mouse, a rat, a dog, a cat, a horse, or a cow. The stem cells may be multipotent, pluripotent, or totipotent cells that can develop.

In the present specification, an excellent (or increased) effect of inducing differentiation into insulin-producing cells may exhibit one or more results of the following characteristics:

(1) An increase in expression levels of a gene and a protein (for example, insulin (INS), pancreas/duodenum homeobox protein 1 (PDX1), V-maf musculoaponeurotic fibrosarcoma oncogene homolog A (MAFA), neurogenin 3 (NEUROG3), neurogenic differentiation 1 (NEUROD1), and/or homeobox protein nkx6.1 (NKX6.1) and the like) involved in beta cell development and maturation in cells;

(2) An increase in an amount of insulin secreted (insulin secretion (μIU/ml)) intracellularly and/or extracellularly by glucose stimulation;

(3) Formation of clusters of islet shape; and

(4) Shortening of the time for differentiation into insulin-producing cells.

It is known that PDX1 is essential for the development of pancreatic exocrine and endocrine cells including beta cells and increases the transcription of the insulin gene, and MAFA binds to the enhancer/promoter region of the insulin gene and binds to glucose to induce insulin expression.

Using conventionally known composition for inducing differentiation or according to previously known differentiation methods (for example, Pagliuca F W et al., 2014 Cell; Rezania A et al., 2014 Nat Biotechnol), it takes about 40 days to differentiate stem cells into insulin-producing cells.

As used herein, “about” or “approximately” may be generally interpreted to mean that a value or range within 20%, 10%, 5%, 4%, 3%, 2%, 1% or 0.5% above and below a given value or range is included.

In an exemplary embodiment, shortening the time for differentiation into insulin-producing cells may mean that it takes 4 days to 10 days, 4 days to 8 days, 3 days to 6 days, 4 days to 6 days, 6 days to 8 days, or 6 days to induce differentiation into cells that secrete insulin in a range of 5 to 25 μIU/ml, 5 to 20 μIU/ml, 5 to 15 μIU/ml, 10 to 25 μIU/ml, 10 to 20 μIU/ml, or 10 to 15 μIU/ml by applying the composition for inducing differentiation according to an exemplary embodiment or by the differentiation method according to an exemplary embodiment.

When cells obtained by inducing differentiation of cells at 5×10⁵ to 5×10⁷ cells/ml, 5×10⁵ to 5×10⁶ cells/ml, or 5×10⁶ to 5×10⁷ cells/ml for 4 days to 10 days, 4 days to 8 days, 3 days to 6 days, 4 days to 6 days, 6 days to 8 days, or 6 days by applying the composition for inducing differentiation (culture medium composition for inducing differentiation) according to an exemplary embodiment or by the differentiation method according to an exemplary embodiment are stimulated with glucose (for example, cultured in a culture medium containing 1 to 30 mM glucose for 1 day to 5 days or 3 days), the cells may secrete insulin at 5 to 25 μIU/ml, 5 to 20 μIU/ml, 5 to 15 μIU/ml, 10 to 25 μIU/ml, 10 to 20 μIU/ml, or 10 to 15 μIU/ml.

An aspect provides a composition for inducing differentiation into insulin-producing cells, comprising putrescine, or

one or more selected from the group consisting of putrescine, glucosamine, and nicotinamide.

In an exemplary embodiment, the composition for inducing differentiation into insulin-producing cells may comprise putrescine.

In an exemplary embodiment, the composition for inducing differentiation into insulin-producing cells may comprise putrescine and glucosamine, or comprise putrescine, glucosamine, and nicotinamide.

According to an exemplary embodiment, the effect of the composition for inducing differentiation into insulin-producing cells may be synergistically increased by a combination of putrescine and glucosamine or a combination of putrescine, glucosamine, and nicotinamide.

The putrescine is “1,4-diaminobutane or 1,4-butanediamine,” and is a type of polyamine found in an entire range of organisms from bacteria to animals and plants, and it is known that putrescine plays an important role in cell proliferation and normal cell growth and is an important material for defense mechanisms against oxidative stress.

According to an exemplary embodiment, among various types of polyamines, putrescine may act as an active material capable of inducing differentiation into insulin-producing cells. The putrescine does not produce toxic metabolites (for example, aldehydes) by polyamine oxidase present in a material (for example, fetal bovine serum (FBS)) which may be used for cell culture, unlike polyamines other than putrescine (for example, spermidine or spermine).

The glucosamine is a type of amino sugar that is a major precursor of biochemical binding between glycoproteins and glycolipids, and forms a part of a polysaccharide structure such as chitosan or chitin, which are the main components constituting the exoskeletons of various arthropods including crustaceans or the cell walls of fungi.

According to an exemplary embodiment, a composition for inducing differentiation (or a culture medium composition for inducing differentiation) comprising putrescine and glucosamine may have a significantly better effect of inducing differentiation into insulin-producing cells than a composition comprising putrescine or glucosamine individually.

According to an exemplary embodiment, in the composition for inducing differentiation (or a culture medium composition for inducing differentiation) comprising putrescine and glucosamine, the concentration of putrescine may be 1 to 20 mM, 1 mM or more to less than 20 mM, 5 to 20 mM, 5 mM or more to less than 20 mM, 1 to 15 mM, 5 to 15 mM, 1 to 10 mM, or 5 to 10 mM.

According to an exemplary embodiment, in the composition for inducing differentiation (or a culture medium composition for inducing differentiation) comprising putrescine and glucosamine, the concentration of glucosamine may be 1 to 20 mM, 1 mM or more to less than 20 mM, 5 to 20 mM, 5 mM or more to less than 20 mM, 1 to 15 mM, 5 to 15 mM, 1 to 10 mM, or 5 to 10 mM.

In an exemplary embodiment, when the concentration of putrescine or glucosamine in the composition for inducing differentiation (or a culture medium composition for inducing differentiation) is more than 20 mM or 20 mM or more, the concentration may not be appropriate for cell differentiation because cell viability may be 70% or less, 60% or less, 50% or less, 40% or less, 37% or less, 5 to 70%, 5 to 60%, 5 to 50%, 5 to 40%, 5 to 37%, 35 to 50%, 35 to 40%, or 36 to 40% when the composition is used for differentiation into insulin-producing cells.

According to an exemplary embodiment, a composition for inducing differentiation (or a culture medium composition for inducing differentiation) comprising putrescine, glucosamine, and nicotinamide may have a significantly better effect of differentiation into insulin-producing cells than a composition comprising putrescine, glucosamine, and/or nicotinamide.

According to an exemplary embodiment, in the composition for inducing differentiation (or a culture medium composition for inducing differentiation) comprising putrescine, glucosamine, and nicotinamide, the concentration of putrescine may be 1 to 20 mM, 1 mM or more to less than 20 mM, 5 to 20 mM, 5 mM or more to less than 20 mM, 1 to 15 mM, 5 to 15 mM, 1 to 10 mM, or 5 to 10 mM.

According to an exemplary embodiment, in the composition for inducing differentiation (or a culture medium composition for inducing differentiation) comprising putrescine, glucosamine, and nicotinamide, the concentration of glucosamine may be 1 to 20 mM, 1 mM or more to less than 20 mM, 5 to 20 mM, 5 mM or more to less than 20 mM, 1 to 15 mM, 5 to 15 mM, 1 to 10 mM, or 5 to 10 mM.

According to an exemplary embodiment, in the composition for inducing differentiation (or a culture medium composition for inducing differentiation), comprising putrescine, glucosamine, and nicotinamide, the concentration of nicotinamide may be 1 to 20 mM, 1 mM or more to less than 20 mM, 5 to 20 mM, 5 mM or more to less than 20 mM, 1 to 15 mM, 5 to 15 mM, 1 to 10 mM, or 5 to 10 mM.

The composition for inducing differentiation into insulin-producing cells according to an exemplary embodiment is safe because genetic manipulation is excluded, and has the advantages that it is capable of inducing differentiation into insulin-producing cells without an expensive compound or growth factor whose mechanism is not clear, conveniently inducing differentiation into insulin-producing cells within a short period of time (for example, within 1 day to 10 days, 1 day to 9 days, 1 day to 8 days, 1 day to 7 days, 1 day to 6 days, 2 days to 10 days, 3 days to 9 days, 4 days to 8 days, 4 days to 6 days, or 6 days), and minimizing contamination, denaturation and the like of cells.

In an exemplary embodiment, when the concentration of putrescine, glucosamine, or nicotinamide in the composition for inducing differentiation (or a culture medium composition for inducing differentiation) is more than 20 mM or 20 mM or more, the concentration may not be appropriate for cell differentiation because cell viability may be 70% or less, 60% or less, 50% or less, 40% or less, 37% or less, 5 to 70%, 5 to 60%, 5 to 50%, 5 to 40%, 5 to 37%, 35 to 50%, 35 to 40%, or 36 to 40% when the composition is used for differentiation into insulin-producing cells.

The composition for inducing differentiation into insulin-producing cells according to an exemplary embodiment may further include a STAT3 inhibitor.

In an exemplary embodiment, the composition for inducing differentiation (or a culture medium composition for inducing differentiation) may further comprise a STAT3 inhibitor on day 0 to day 8, day 2 to day 6, day 3 to day 5, day 2 to day 4, day 3 to day 4, day 4 to day 5, or day 4 after a culture begins. According to an exemplary embodiment, when the composition for inducing differentiation further includes the STAT3 inhibitor at a period of time in the above-described range, the effect of the composition for inducing differentiation (or a culture medium composition for inducing differentiation) of inducing differentiation into insulin-producing cells may be significantly increased.

According to an exemplary embodiment, a composition for inducing differentiation (or a culture medium composition for inducing differentiation) comprising putrescine, glucosamine, nicotinamide, and a STAT3 inhibitor may have a significantly better effect of differentiation into insulin-producing cells than a composition comprising putrescine, glucosamine, nicotinamide, and/or a STAT3 inhibitor.

According to an exemplary embodiment, the concentration of putrescine in the composition for inducing differentiation (or a culture medium composition for inducing differentiation) comprising putrescine, glucosamine, nicotinamide, and a STAT3 inhibitor may be 1 to 20 mM, 1 mM or more to less than 20 mM, 5 to 20 mM, 5 mM or more to less than 20 mM, 1 to 15 mM, 5 to 15 mM, 1 to 10 mM, or 5 to 10 mM.

According to an exemplary embodiment, in the composition for inducing differentiation (or a culture medium composition for inducing differentiation) comprising putrescine, glucosamine, nicotinamide, and a STAT3 inhibitor, the concentration of glucosamine may be 1 to 20 mM, 1 mM or more to less than 20 mM, 5 to 20 mM, 5 mM or more to less than 20 mM, 1 to 15 mM, 5 to 15 mM, 1 to 10 mM, or 5 to 10 mM.

According to an exemplary embodiment, the concentration of nicotinamide in the composition for inducing differentiation (or a culture medium composition for inducing differentiation) comprising putrescine, glucosamine, nicotinamide, and a STAT3 inhibitor may be 1 to 20 mM, 1 mM or more to less than 20 mM, 5 to 20 mM, 5 mM or more to less than 20 mM, 1 to 15 mM, 5 to 15 mM, 1 to 10 mM, or 5 to 10 mM.

According to an exemplary embodiment, the concentration of a STAT3 inhibitor in the composition for inducing differentiation (or a culture medium composition for inducing differentiation) comprising putrescine, glucosamine, nicotinamide, and the STAT3 inhibitor may be 0.01 to 100 μM, 0.1 to 100 μM, 0.1 to 50 μM, 0.5 to 50 μM, 0.1 to 20 μM, 0.1 to 15 μM, 0.5 to 15 μM, 0.5 to 10 μM, 0.1 to 10 μM, 1 to 20 μM, 1 to 15 μM, or 1 to 10 μM.

Signal transducer and transcriptions (STAT) are transcription factors with seven subunit forms: STAT1, STAT2, STAT3, STAT4, STAT5a, STAT5b, and STAT6, and in particular, STAT3 is a transcriptional regulator that is continuously activated through various pathways and serves to promote tumorigenesis, is involved in the transcription of various genes in cells, and responds to external signaling by cytokines or growth factors in normal cases, and as a result, STAT3, present in cytoplasm, migrates into the nucleus to regulate genes for cell development, differentiation, growth, survival, angiogenesis, immune functions, and the like.

In an exemplary embodiment, the STAT3 inhibitor may further include one or more selected from the group consisting of JSI-124, BP-1-102, CPT, 8-hydroxy-3-methyl-3,4-dihydrotetraphene-1,7,12(2H)-trione (STA-21), (E)-2-cyano-3-(3,4-dihydrophenyl)-N-(phenylmethyl)-2-propenamide (AG-490), 3-(8,8-dipropyl-3-azaspiro[4.5]decan-3-yl)-N,N-diethylpropan-1-amine (atiprimod), 3,4,5-triacetyloxy-6-(acetyloxymethyl) oxane-2-thiolate (auranofin; triethylphosphanium), sodium 2-(auriosulfanyl)-3-carboxypropanoate (aurothiomalate), N-(2-chloro-6-methylphenyl)-2-[[6-[4-(2-hydroxyethyl)-1-piperazinyl]-2-methyl-4 pyrimidinyl]amino]-5-thiazole carboxamide monohydrate (BMS-354825; dasatinib), 3-(3,4-dihydroxy-phenyl)-acrylic acid 2-(3,4-dihydroxy-phenyl)-ethyl ester (CADPE), 6-nitro-1,1-dioxide-benzo[b]thiophene (Stattic), calcium 2,5-dihydroxybenzenesulfonate (dobesilate), ethanol, NCX-4016 (NO-aspirin), (3S,4aS,8aS)—N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide (nelfinavir), phosphododecapeptide (PDP), (1R)-3-methyl-1-({(2S)-3-phenyl-2-[(pyrazin-2-ylcarbonyl)amino]propanoyl}amino)butyl]boronic acid (PS-341; bortezomib), 6-[amino(4-chlorophenyl)(1-methyl-1H-imidazol-5-yl)methyl]-4-(3-chlorophenyl)-1-methylquinolin-2(1H)-one (R115777; tipifarnib), N-hexyl-2-(1-naphthalenyl)-5-[[4-(phosphonooxy)phenyl] (S31-M2001), a statin-based compound, sodium salicylate, 3-(3,4-dihydroxyphenyl)-2-propenoic acid 3,4-dihydroxy-cinnamic acid trans-caffeate 3,4-dihydroxytrans-cinnamate (caffeic acid), 1,8-dihydroxy-3-(hydroxymethyl)-9,10-anthracenedione (emodin), 3-beta-3-hydroxy-urs-12-ene-28-oic-acid (ursolic acid), 3,5,4′-trihydroxy-trans-stilbene (resveratrol), (2E,4E,6E,8E)-3,7-dimethyl-9-(2,6,6-trimethylcyclohexen-1-yl)nona-2,4,6,8-tetraenoic acid (retinoic acid), (2R,3R)-5,7-dihydroxy-2-(3,4,5-trihydroxyphenyl)chroman-3-yl] 3,4,5-trihydroxybenzoate (epigallocatechingallate (EGCG)), (E,E,Z,E)-3-methyl-7-(4-methylphenyl)-9-(2,6,6-trimethyl-1-cyclohexen-1-yl)-2,4,6,8-nonatetraenoic acid (Sr11302), 1,6,6-trimethyl-6,7,8,9-tetrahydrophenanthro[1,2-b]furan-10,11-dione (tanshinone IIA), and (1E,6E)-1,7-bis(4-hydroxy-3-methoxyphenyl)-1,6-heptadiene-3,5-dione (curcumin).

According to an exemplary embodiment, the STAT3 inhibitor may be added after being dissolved in an organic solvent, and the organic solvent may be used without limitation as long as it can be typically used, and may be for example, DMSO, DMF, acetonitrile, methanol, ethanol, isopropyl alcohol, water, dichloromethane, THF, and/or ethyl acetate.

The BP-1-102 (Cas No. 1334493-07-0) may have a chemical formula of the following Chemical Formula 1, and according to an exemplary embodiment, when BP-1-102 among the STAT3 inhibitors is further comprised in the composition for inducing differentiation into insulin-producing cells, the effect of inducing differentiation into insulin-producing cells may be better than that when other STAT3 inhibitors (for example, JSI-124 and/or CPT) are added. According to an exemplary embodiment, the BP-1-102 may be added thereto after being dissolved in an organic solvent, and the concentration of BP-1-102 in the composition for inducing differentiation may be 0.1 to 100 μM, 0.1 to 50 μM, 1 to 50 μM, 5 to 50 μM, 5 to 20 μM, 1 to 10 μM, 5 to 10 μM, 5 to 15 μM, 10 to 20 μM, 10 to 15 μM, or 10 μM.

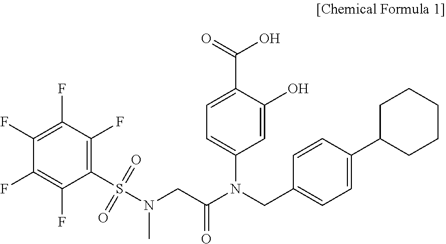 [SMILES: CN(CC(=O)N(Cc1ccc(C2CCCCC2)cc1)c1ccc(C(=O)O)c(O)c1)S(=O)(=O)c1c(F)c(F)c(F)c(F)c1F]

The JSI-124 (Cas No. 2222-07-3) may have a chemical formula of the following Chemical Formula 2, and according to an exemplary embodiment, when JSI-124 among the STAT3 inhibitors is further comprised in the composition for inducing differentiation into insulin-producing cells, the effect of inducing differentiation into insulin-producing cells may be better than that when other STAT3 inhibitors (for example, CPT) are added. According to an exemplary embodiment, the JSI-124 may be added thereto after being dissolved in an organic solvent, and the concentration of JSI-124 in the composition for inducing differentiation may be 0.01 to 100 μM, 0.1 to 100 μM, 0.1 to 50 μM, 0.1 to 30 μM, 0.1 to 20 μM, 0.1 to 15 μM, 0.1 to 10 μM, 0.1 to 5 μM, 0.1 to 1 μM, or 0.1 μM.

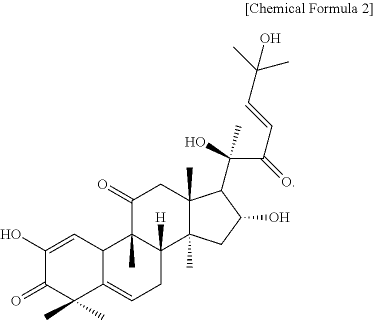

In an exemplary embodiment, the composition for inducing differentiation into insulin-producing cells may induce the differentiation of one or more cells selected from the group consisting of adult stem cells, embryonic stem cells, induced pluripotent stem cells (iPS cells), and progenitor cells into insulin-producing cells.

The cells, for example, adult stem cells, embryonic stem cells, induced pluripotent stem cells (iPS cells), or progenitor cells, may be derived from a mammal such as a human, a non-human primate, and/or a mouse, a rat, a dog, a cat, a horse, or a cow.

Since the composition for inducing differentiation, the culture medium composition for inducing differentiation, and/or the differentiation method according to an exemplary embodiment may induce differentiation from various types of cells into insulin-producing cells, the differentiation from cells that are easy to obtain (for example, umbilical cord blood cells or bone marrow cells) into insulin-producing cells may be efficiently induced without being limited to types of cells, thereby enabling insulin-producing cells to be conveniently mass-produced.

Embryonic stem cells are stem cells derived from fertilized eggs and have a property of being able to differentiate into cells of all tissues.

Induced pluripotent stem cells (iPS cells) are also called dedifferentiated stem cells, and are cells that have been induced to be pluripotent, like embryonic stem cells, by injecting cell-differentiation-related genes into somatic cells that have completed differentiation and returning the somatic cells to the cell stage prior to differentiation.

Progenitor cells have the ability to differentiate into specific types of cells similar to stem cells, but are more specific and targeted than stem cells, and unlike stem cells, the number of divisions is limited. The progenitor cells may be mesenchymal-derived progenitor cells, but are not limited thereto. In the present specification, progenitor cells are included in the category of stem cells, and unless otherwise specified, ‘stem cells’ is interpreted as a concept that also includes progenitor cells.

Adult stem cells are stem cells extracted from the umbilical cord, umbilical cord blood, or adult bone marrow, blood, nerves, skin, fat, and the like, and are primitive cells on the cusp of differentiating into cells of specific organs. The adult stem cell may be one or more selected from the group consisting of a hematopoietic stem cell, a mesenchymal stem cell, a neural stem cell, and the like. Adult stem cells have characteristics of being capable of not only regenerating various organs required in actual medicine using various types of adult stem cells, but also differentiating to be suitable for the characteristics of each organ, instead of being difficult to proliferate and having a strong tendency to differentiate easily, and thus may be advantageously applied to the treatment of intractable/incurable diseases.

In an exemplary embodiment, the adult stem cells may be mesenchymal stem cells (MSCs). Mesenchymal stem cells are also called mesenchymal stromal cells (MSCs), and are multipotent stromal cells capable of differentiating into various forms of cells such as osteoblasts, chondrocytes, myocytes, and adipocytes. Mesenchymal stem cells may be selected among multipotent cells derived from non-marrow tissues such as placenta, umbilical cord, umbilical cord blood, adipose tissue, adult muscle, corneal stroma, and deciduous dental pulp.

In an exemplary embodiment, the mesenchymal stem cells may be human embryonic stem cell-derived mesenchymal stem cells (ES-MSCs).

As used herein, a culture medium is something that is able to support the induction of differentiation into insulin-producing cells and the survival of cells in vitro, and all typical culture media used in the art suitable for culturing cells are included. According to the type of cell, culture media and culture conditions may be selected. In an exemplary embodiment, the culture medium used for the culture is a cell culture minimum medium (CCMM), and may generally include a carbon source, a nitrogen source, and a trace element component.

In an exemplary embodiment, the composition for inducing differentiation into insulin-producing cells may further include one or more culture media selected from the group consisting of CMRL1066, a Dulbecco's Modified Eagle's Medium (DMEM), a minimal essential medium (MEM), Basal Medium Eagle (BME), RPMI1640, F-10, F-12, an α minimal essential medium (αMEM), a Glasgow's minimal essential medium (GMEM), McCoy's 5A, a 199 medium, and endothelial growth medium MV2.

Further, the medium may be a serum-free medium depending on the cell type and/or purpose, or may further include serum (for example, fetal bovine serum (FBS), bovine calf serum (BCS), horse serum, human serum, and the like) and/or an antibiotic (for example, penicillin, streptomycin, gentamicin, and the like) as a supply source of growth factors.

The CMRL1066 culture medium is a culture medium that is rich in glycosylamines, nucleosides, vitamins, and growth factors, is a culture medium that is widely used in cell culture, and has not been used as a culture medium for differentiation into insulin-producing cells in the related art.

In an exemplary embodiment, the CMRL1066 culture medium may include 10% FBS.

In an exemplary embodiment, the CMRL1066 culture medium may include glucose, FBS, and an antibiotic.

In an exemplary embodiment, the CMRL1066 culture medium includes D-glucose at about 1000 mg/L, phenol red at about 20 mg/L, and sodium bicarbonate at about 2200 mg/L, and may have a pH of 7.3±0.3. As the CMRL1066 culture medium, one that is commercially available may be used.

In an exemplary embodiment, the DMEM culture medium may include glucose, FBS, and an antibiotic. As the DMEM culture medium, one that is commercially available may be used, and for example, the DMEM culture medium may be manufactured by Hyclone, and may be one with catalogue No. SH30021, SH30022, or SH30307. The DMEM culture medium may include D-glucose at about 1000 mg/L, phenol red at about 15 to 16 mg/L, and sodium bicarbonate at about 3700 mg/L.

In an exemplary embodiment, the composition for inducing differentiation into insulin-producing cells may further include one or more conventionally known materials that induce differentiation into insulin-producing cells.

Another aspect provides a culture medium composition for inducing differentiation into insulin-producing cells, comprising the composition for inducing differentiation into insulin-producing cells and a culture medium.

In an exemplary embodiment, the culture medium may be one or more media selected from the group consisting of CMRL1066, a Dulbecco's Modified Eagle's Medium (DMEM), a minimal essential medium (MEM), Basal Medium Eagle (BME), RPMI1640, F-10, F-12, an α minimal essential medium (αMEM), a Glasgow's minimal essential medium (GMEM), McCoy's 5A, a 199 medium, and endothelial growth medium MV2. Details on the culture medium are as described above.

According to an exemplary embodiment, the culture medium composition for inducing differentiation into insulin-producing cells, comprising the composition comprising putrescine, glucosamine, and nicotinamide and the CMRL1066 culture medium, may have a significantly better effect of inducing differentiation into insulin-producing cells than a culture medium composition comprising a different type of culture medium.

In an exemplary embodiment, the culture medium composition for inducing differentiation into insulin-producing cells may further comprise one or more conventionally known materials that induce differentiation into insulin-producing cells.

In an exemplary embodiment, a composition for inducing differentiation (or a culture medium composition for inducing differentiation) consisting of putrescine, glucosamine, nicotinamide, a STAT3 inhibitor (for example, JSI-124, BP-1-102, or CPT), and CMRL1066 may have an excellent effect of inducing differentiation from stem cells into insulin-producing cells even without comprising any other active ingredient.

Still another aspect provides a method for inducing differentiation into insulin-producing cells, the method comprising culturing isolated cells in the culture medium composition for inducing differentiation (or the composition for inducing differentiation).

In the culturing step, the cells may be cultured adherently and/or in suspension.

In an exemplary embodiment, the culturing step may comprise maintaining optimal conditions for differentiation into insulin-producing cells.

According to an exemplary embodiment, the culturing step may comprise culturing isolated cells in the culture medium composition under conditions of a temperature of 35 to 38° C. and 5% CO₂ for 3 days to 6 days.

According to an exemplary embodiment, the culturing step may comprise seeding cells into a plate comprising the culture medium composition at a concentration of 0.5×10⁶ cells/ml to 10⁷ cells/ml, 0.5×10⁶ cells/ml to 5×10⁶ cells/ml, 5×10⁶ cells/ml to 10⁷ cells/ml or 5×10⁶ cells/ml, and culturing the seeded cells under conditions of a temperature of 25 to 40° C., 28 to 38° C., 25 to 35° C., 35 to 38° C., 35 to 37° C. or 37° C. and a pH of 7.0 to 7.5, 7.1 to 7.4, or 7.3 for 1 day to 14 days, 1 day to 12 days, 1 day to 10 days, 1 day to 8 days, 1 day to 7 days, 1 day to 6 days, 2 days to 14 days, 4 days to 14 days, 6 days to 14 days, 2 days to 10 days, 3 days to 9 days, 3 days to 8 days, 3 days to 6 days, 4 days to 6 days, or 6 days.

According to an exemplary embodiment, the culture medium composition comprising cells isolated in the culturing step may be stirred at a rate of 10 to 100 rpm, 10 to 80 rpm, 30 to 80 rpm, 80 rpm, or 30 to 60 rpm.

Air-enriched with 5% CO₂ may be used, or a carbonate buffer solution may be used to maintain the above-mentioned pH range.

The culture medium in the culture medium composition may be one or more culture media selected from the group consisting of CMRL1066, a Dulbecco's Modified Eagle's Medium (DMEM), a minimal essential medium (MEM), Basal Medium Eagle (BME), RPMI1640, F-10, F-12, an α minimal essential medium (αMEM), a Glasgow's minimal essential medium (GMEM), McCoy's 5A, a 199 medium, and endothelial growth medium MV2. Details on the culture medium are as described above.

According to an exemplary embodiment, when cells are cultured in a culture medium composition comprising a composition including putrescine, glucosamine, and nicotinamide and the CMRL1066 culture medium, the effect of inducing differentiation into insulin-producing cells may be significantly better than that in cells cultured in a culture medium composition comprising a culture medium different from the CMRL1066 medium.

In an exemplary embodiment, the method for inducing differentiation into insulin-producing cells may increase the mRNA or protein expression of beta cell-related factors about 1- to 500-fold, 1- to 100-fold, 10- to 50-fold, or 1- to 10-fold when the cells in the culturing step are cultured for 1 day to 14 days, 1 day to 12 days, 1 day to 10 days, 1 day to 8 days, 1 day to 7 days, 1 day to 6 days, 2 days to 14 days, 4 days to 14 days, 6 days to 14 days, 2 days to 10 days, 3 days to 9 days, 4 days to 8 days, 4 days to 6 days, or 6 days, compared to the control in which cells are cultured in a culture medium composition which does not comprise the composition for inducing differentiation according to an exemplary embodiment.

In an exemplary embodiment, the method for inducing differentiation into insulin-producing cells may comprise culturing cells by adding the STAT3 inhibitor to the culture medium composition on day 2 to day 6, day 3 to day 5, day 2 to day 4, day 3 to day 4, day 4 to day 5, or day 4 after the cell culture. When the STAT3 inhibitor is added at a period of time within the above range, the effect of inducing differentiation into insulin-producing cells may be better than the effect when the STAT3 inhibitor is added at a period of time outside of the above range.

In an exemplary embodiment, cells (isolated cells) are cultured in a culture medium composition comprising putrescine, glucosamine, and/or nicotinamide, and may be cultured by adding a STAT3 inhibitor (for example, BP-1-102 and/or JSI-124) on day 2 to day 6, day 3 to day 5, day 2 to day 4, day 3 to day 4, day 4 to day 5, or day 4 after the culture.

In an exemplary embodiment, the STAT3 inhibitor may be one or more selected from the group consisting of JSI-124, BP-1-102, and CPT. Details on the STAT3 inhibitor are as described above.

In an exemplary embodiment, the cells may be one or more selected from the group consisting of adult stem cells, embryonic stem cells, induced pluripotent stem cells (iPS cells), and progenitor cells. Details on the adult stem cells, embryonic stem cells, induced pluripotent stem cells (dedifferentiated pluripotent stem cells), and progenitor cells are as described above.

Yet another aspect provides a cell therapeutic agent for treating diabetes mellitus, comprising insulin-producing cells differentiated by the method for inducing differentiation into insulin-producing cells.

As used herein, ‘cell therapeutic agent’ refers to pharmaceuticals used for the purpose of treatment, diagnosis, and prevention, by using a cell or tissue prepared through isolation, culture and specific manipulation from humans (US FDA regulations), and specifically, it refers to a drug used for the purpose of treatment, diagnosis, and prevention through a series of actions of in vitro proliferating and sorting living autologous, allogenic or xenogeneic cells, or changing the biological characteristics of cells in other ways to restore the function of cells or tissues. Cell therapeutic agents may be broadly classified into somatic cell therapeutic agents and stem cell therapeutic agents depending on the degree of cell differentiation.

As used herein, “diabetes mellitus” refers to a metabolic disease such as insufficient secretion of insulin or malfunction of insulin.

Yet another aspect provides a therapeutic composition for preventing or treating diabetes mellitus, comprising a composition for inducing differentiation and a mixture of patient-derived blood.

The patient-derived blood may be taken and isolated from the patient.

The patient-derived blood may include patient-derived cells, and may include one or more cells selected from the group consisting of patient-derived adult stem cells, embryonic stem cells, induced pluripotent stem cells (iPS cells), and progenitor cells. The pharmaceutical composition for preventing or treating diabetes mellitus may include insulin-producing cells in which patient-derived cells included in blood are differentiated by the composition for inducing differentiation.

The pharmaceutical composition for preventing or treating diabetes mellitus may be applied to a patient from whose blood has been isolated to prevent or minimize the immune response, and thus may be efficiently used for the purpose of preventing or treating diabetes mellitus.

The patient may be a subject at risk of developing diabetes mellitus or have been diagnosed with diabetes, and the subject may be any mammals that may develop diabetes mellitus, for example, a human or a primate, as well as a domestic animal such as a cow, a pig, a sheep, a horse, a dog, or a cat.

Yet another aspect provides a pharmaceutical composition for preventing or treating diabetes mellitus, comprising insulin-producing cells differentiated by the method for inducing differentiation into insulin-producing cells.

The diabetes mellitus may be type 1 diabetes mellitus or type 2 diabetes mellitus.

Further, the present invention provides a method for preventing or treating diabetes mellitus, the method comprising administering a composition comprising the composition for inducing differentiation of the present invention and a mixture of patient-derived blood as an active ingredient to a subject in need.

In addition, the present invention provides a use of a composition for inducing differentiation of the present invention and a mixture of patient-derived blood as an active ingredient for preventing or treating diabetes mellitus.

Furthermore, the present invention provides a method for preventing or treating diabetes mellitus, the method comprising administering a composition comprising the insulin-producing cells differentiated by the method of the present invention as an active ingredient to a subject in need.

Further, the present invention provides a use of a composition comprising the insulin-producing cells differentiated by the method of the present invention as an active ingredient for preventing or treating diabetes mellitus.

As used herein, “administration” refers to provision of a predetermined substance to a patient by any appropriate method, and as the route of administration of a pharmaceutical composition, the composition of the present invention may be administered via any general route that allows the drug to reach a target tissue. The route of administration may be intraperitoneal administration, intravenous administration, intramuscular administration, subcutaneous administration, intradermal administration, oral administration, topical administration, intranasal administration, intrapulmonary administration, rectal administration, and the like, but is not limited thereto. However, since the peptide is digested during oral administration, it is preferable for the oral composition to be formulated with an active agent coated or otherwise protected from gastric degradation. Preferably, the composition of the present invention may be administered in the form of an injection. Further, a long-acting agent may be administered by any device capable of transferring the active material to a target cell.

According to an exemplary embodiment, the pharmaceutical composition may include a pharmaceutically acceptable carrier. As used herein, “pharmaceutically acceptable carrier” refers to a carrier or diluent that does not stimulate an organism and does not inhibit the biological activity or properties of an administered compound. When the pharmaceutically acceptable carrier is orally administered, a binder, a lubricant, a disintegrant, an excipient, a solubilizing agent, a dispersing agent, a stabilizer, a suspending agent, a colorant, a flavoring agent, and the like may be used, in the case of injection, a buffering agent, a preservative, an analgesic, a solubilizer, an isotonic agent, a stabilizer, and the like may be mixed and used, and in the case of topical administration, a base, an excipient, a lubricant, a preservative, and the like may be used. The formulation of the pharmaceutical composition of the present invention may be variously prepared by mixing the pharmaceutical composition of the present invention with the pharmaceutically acceptable carrier as described above. For example, the formulation may be prepared in the form of a tablet, a troche, a capsule, an elixir, a suspension, a syrup, a wafer, and the like when orally administered, and in the case of injection, the injection may be formulated into unit dosage ampoules or in multiple dosage forms. The pharmaceutical composition of the present invention may be formulated into other solutions, suspensions, globules, capsules, sustained-release preparations, and the like.

Meanwhile, as an example of suitable carriers, excipients and diluents for formulation, it is possible to use lactose, dextrose, sucrose, sorbitol, mannitol, xylitol, erythritol, maltitol, starch, acacia, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oil, or the like. Further, the pharmaceutical composition of the present invention may additionally include a filler, an anticoagulant, a lubricant, a wetting agent, a flavoring agent, an antiseptic, and the like.

The pharmaceutical composition may additionally include a pharmaceutically acceptable carrier, and the carrier is typically used in formulations and includes, for example, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, and the like, but is not limited thereto. The pharmaceutical composition may additionally include a lubricant, a wetting agent, a sweetening agent, a flavoring agent, an emulsifier, a suspending agent, a preservative, and the like, in addition to the aforementioned ingredients. Suitable pharmaceutically acceptable carriers and formulations are described in detail in Remington's Pharmaceutical Sciences (19th ed., 1995).

The pharmaceutical composition may be administered by being formulated as a unit-administered pharmaceutical preparation suitable for administration into the body of a patient by a typical method in the pharmaceutical field, and the preparation includes an effective dose by administration once or several times. As a formulation suitable for such a purpose, an injection, an infusion, or the like is preferred as a parenteral preparation. In addition, the pharmaceutical composition may be transplanted and administered using an administration method typically used in the art, and preferably can be directly engrafted or transplanted to a diseased site of a patient in need of treatment, but is not limited thereto. Furthermore, all of the administrations can be performed by non-surgical administration using a catheter and surgical administration methods such as injection or transplantation after incision of a diseased site. As a dose, 1.0×10⁴ to 1.0×10¹⁰ cells/kg body weight, preferably 1.0×10⁵ to 1.0×10⁹ cells/kg body weight may be administered once or divided into several doses. However, it is to be understood that the actual dose of the active ingredient needs to be determined in consideration of various related factors such as the disease to be treated, the severity of the disease, the administration route, the body weight, age and sex of a patient, and accordingly, the dose does not limit the scope of the present invention in any way.

The pharmaceutical composition for preventing or treating diabetes mellitus according to an exemplary embodiment may be used as a new cell therapeutic agent for diabetes mellitus, which solves the side effect and stability problems of existing therapeutic agents for diabetes mellitus.

### Advantageous Effects

By using a differentiation inducing composition according to an exemplary embodiment or a differentiation inducing method according to an exemplary embodiment, insulin-producing cells can be prepared in a short period by effectively inducing the differentiation of various types of stem cells into insulin-producing cells, and can be mass-produced in a relatively simple manner, and thus a pharmaceutical composition for preventing or treating diabetes mellitus, comprising insulin-producing cells and/or insulin produced thereby, can be provided.

### DESCRIPTION OF DRAWINGS

FIG. 1A illustrates a schematic view of the experimental procedure for transplantation of bone marrow cells treated (or primed) with putrescine into diabetes mellitus mouse models prepared by streptozotocin (STZ) administration, and FIG. 1B illustrates the results of measuring random blood glucose (upper graph) and body weight (lower graph) of the diabetes mellitus mouse models. In FIG. 1B, the control indicates normal mice in which diabetes mellitus has not been induced, (STZ) indicates diabetes mellitus mouse models, (STZ+control cells) indicates diabetes mellitus mouse models transplanted with bone marrow cells in which differentiation has not been induced, and (STZ+Put-primed cells) indicates diabetes mellitus mouse models transplanted with bone marrow cells primed with putrescine.

FIG. 2A illustrates the mRNA expression levels of insulin (INS) and factors that promote the differentiation of insulin-producing beta cells (MAFA, PDX-1, and NEUROG3) in human bone marrow cells that have been induced to differentiate by adding putrescine, and FIG. 2B illustrates the mRNA expression levels of insulin (INS) and factors that promote the differentiation of insulin-producing beta cells (MAFA, NEUROG3) in umbilical cord blood cells that have been induced to differentiate by adding putrescine. In FIG. 2A, * means p<0.05; and ** means p<0.01, and the same applies to the following drawings.

FIG. 3 illustrates the mRNA expression levels of insulin (INS) and beta cell differentiation-related genes (MAFA, PDX1, or NEUROG3) in cells, in which mouse bone marrow cells were treated with glucosamine and/or putrescine to induce differentiation. In FIG. 3, ‘GlcN’ means glucosamine, ‘Put’ means putrescine, ‘−’ means that the corresponding component is not included, and ‘+’ means that the corresponding component is included. The same applies to other drawings.

FIG. 4 illustrates the mRNA expression levels of insulin (INS) and beta cell differentiation-related genes (MAFA, PDX1, or NEUROG3) in cells, in which umbilical cord blood cells were treated with glucosamine, putrescine, and/or nicotinamide to induce differentiation. In FIG. 4, ‘GlcN’ means glucosamine, ‘Put’ means putrescine, NAD means nicotinamide, and the same applies to other drawings.

FIG. 5 illustrates the mRNA expression levels of insulin (INS) and a beta cell differentiation-related gene (PDX1) in cells, in which mouse bone marrow cells were treated with glucosamine, putrescine, and/or nicotinamide to induce differentiation.

FIG. 6A and FIG. 6B illustrate the mRNA expression levels of insulin (INS) and beta cell differentiation-related genes (MAFA, PDX1, NEUROG3, NEUROD1, or NKX6.1) in cells, in which umbilical cord blood cells were treated with glucosamine, putrescine, and nicotinamide in a DMEM or CMRL1066 culture medium to induce differentiation. Specifically, FIG. 6A illustrates the results of RT-PCR, and FIG. 6B illustrates the results of quantitative real-time PCR for each gene. In FIGS. 6A and 6B, NIT-1 means an insulin-producing cell line (beta cell line; insulinoma) in which differentiation has been completed, and the same applies to the following drawings. In FIGS. 6A and 6B, ‘−’ means the results for cells cultured in a culture medium which does not include glucosamine, putrescine, or nicotinamide, and ‘P+G+N’ or ‘P/G/N’ means the results in cells in which differentiation has been induced in a culture medium including glucosamine, putrescine, and nicotinamide.

FIG. 7A illustrates a method of inducing differentiation into insulin-producing cells by co-treating human embryonic mesenchymal stem cells with glucosamine, putrescine, and nicotinamide. FIG. 7B illustrates the results (RT-PCR) of comparing the expression levels of insulin and beta cell differentiation-related genes (PDX1, NEUROG3, NEUROD1, or NKX6.1) for cells in which embryonic mesenchymal stem cells have been treated with glucosamine, putrescine, and nicotinamide together to induce differentiation. In FIG. 7B, CTL means the control cultured in CMRL 1066 culture medium which does not include glucosamine, putrescine, or nicotinamide, and the numbers 1, 2, and 3 represent the results of repeated experiments under the same conditions, respectively.

FIG. 8A illustrates a schematic view of a method of inducing differentiation into insulin-producing cells by treating mouse bone marrow cells with glucosamine, putrescine, and nicotinamide together to culture cells, and treating the cells with a STAT3 inhibitor JSI-124 on day 0 (at the start of culture), day 2 or day 4 after the culture. FIGS. 8B and 8C illustrate the results of comparing the expression levels of insulin and beta cell differentiation-related genes (PDX1, NEUROG3, or NEUROD1) for cells that have been induced to differentiate into insulin-producing cells by treating bone marrow cells with glucosamine, putrescine, and nicotinamide together to prime cells and treating the cells with a STAT3 inhibitor JSI-124 on day 0, 2, or 4 after the culture. Specifically, FIG. 8B illustrates RT-PCR results, FIG. 8C illustrates quantitative real-time PCR results, and in FIG. 8C, ‘-’ means a group which is treated with only putrescine, glucosamine, and nicotinamide, and is not treated with a STAT3 inhibitor.

FIG. 9A illustrates a schematic view of a method of inducing differentiation into insulin-producing cells by treating umbilical cord blood cells with glucosamine, putrescine, and nicotinamide together to culture cells and further treating the cells with various types of STAT3 inhibitors (JSI-124, CPT, or BP-1-102) on day 4 after the culture. FIG. 9B illustrates the results of comparing the expression levels of insulin and beta cell differentiation-related genes (MAFA, PDX1, NEUROG3, NEUROD1, or NKX6.1) for cells that have been induced to differentiate into insulin-producing cells by treating umbilical cord blood cells with glucosamine, putrescine, and nicotinamide together to culture cells and treating the cells with various types of STAT3 inhibitors (JSI-124, CPT, or BP-1-102) on day 4 after the culture (FIG. 9B does not illustrate the results for CPT).

FIG. 10A illustrates the results of a method of inducing differentiation into insulin-producing cells by treating umbilical cord blood cells with glucosamine, putrescine, and nicotinamide together to culture cells, treating the cells with BP-1-102 on day 4 after the culture, and then maintaining the cells in suspension for 6 days to induce differentiation into insulin-producing cells. Subsequently, the cells were washed and re-suspended in CMRL 1066 culture medium, and then cultured for an additional 3 days. FIG. 10B illustrates the results of measuring the insulin secretion in cells that have been induced to differentiate by the method described in FIG. 10A.

FIG. 11A illustrates the efficacy of inducing differentiation into insulin-producing cells by the different concentrations of putrescine in the composition for inducing differentiation. FIG. 11B illustrates the cell viability (%) by the different concentrations of putrescine in the composition for inducing differentiation.

FIG. 12A illustrates a schematic view of a method of transplanting bone marrow cells primed with putrescine, glucosamine, nicotinamide and a STAT3 inhibitor into a kidney capsule of mice on day 7 after STZ induction to generate a diabetes mellitus mouse model. In FIG. 12A, BMNCs indicate bone marrow-derived mononuclear cells (referred to as BMNCs or bone marrow cells herein). FIGS. 12B to 12D illustrate the results of measuring food and water intake, random blood glucose, and body weight of the diabetes mellitus mouse models transplanted with the primed BMNCs for 42 days after STZ induction. FIG. 12E illustrates the results of glucose tolerance test. The mice were fasted for 16 hours and intraperitoneally injected with a bolus of glucose (1 mg/g body weight) and the blood glucose levels were measured at the indicated time points. FIG. 12F illustrates the results of in vivo glucose-stimulated insulin secretion that measures the insulin levels in blood after glucose stimulation of 2 mg/g body weight in a diabetes mellitus mouse model transplanted with the primed BMNCs.

FIG. 13A illustrates the results of observing the protein expression for insulin and PDX1 by immunofluorescence staining in diabetes mellitus mouse models transplanted with BMNCs primed with putrescine, glucosamine, nicotinamide and a STAT3 inhibitor. FIG. 13B illustrates the results of observing the expression of insulin protein through immunohistochemistry with anti-insulin antibody after isolating pancreatic tissue from a diabetes mellitus mouse model transplanted with BMNCs-derived insulin-producing cells.

### MODES OF THE INVENTION

The present invention will be described in more detail with reference to the following examples, but the scope of rights is not intended to be limited to the following examples.

### Example 1. Induction of Differentiation into Insulin-Producing Cells by Putrescine

### Example 1.1 Effect of Reducing Blood Glucose in a Diabetes Mellitus Mouse Model

In the present example, it was intended to investigate whether the diabetes mellitus mouse models transplanted with bone marrow cells primed with putrescine had the ability to regulate blood glucose levels.

Bone marrow cells were prepared by flushing the femurs and tibias of 8-week-old male C57B/6 mice (Seoul National University Institute of Laboratory Animal Resources). Whole bone marrow cells were suspended in a lysis solution (BD Pharmingen) to remove red blood cells, washed, and re-suspended in the standard culture medium (Dulbecco's Modified Eagle's Medium (DMEM)) supplemented with 5.5 mM glucose, 10% FBS, and 1% antibiotics. Bone marrow cells (5×10⁶ cells/well) were seeded in a 12-well non-coated plate, treated with putrescine to a final concentration of 10 mM, and cultured for 6 days in suspension on a shaking (30-60 rpm) platform in a cell culture incubator (37° C., 5% CO₂, and 90 to 95% humidity).

Matrigel grafts were prepared by mixing bone marrow cells primed with putrescine (2×10⁶ cells) with the same volume of Matrigel (BD), and transplanted into the subcutaneous space on the back of mice. Diabetes mellitus was induced in 8-week-old male C57BL/6 mice (Orientbio Inc.) by a single intraperitoneal injection of 140 mg/kg streptozotocin (STZ, Sigma) and the mice with random blood glucose level at 400 mg/dl or more were considered as STZ-induced diabetes mellitus mouse models.

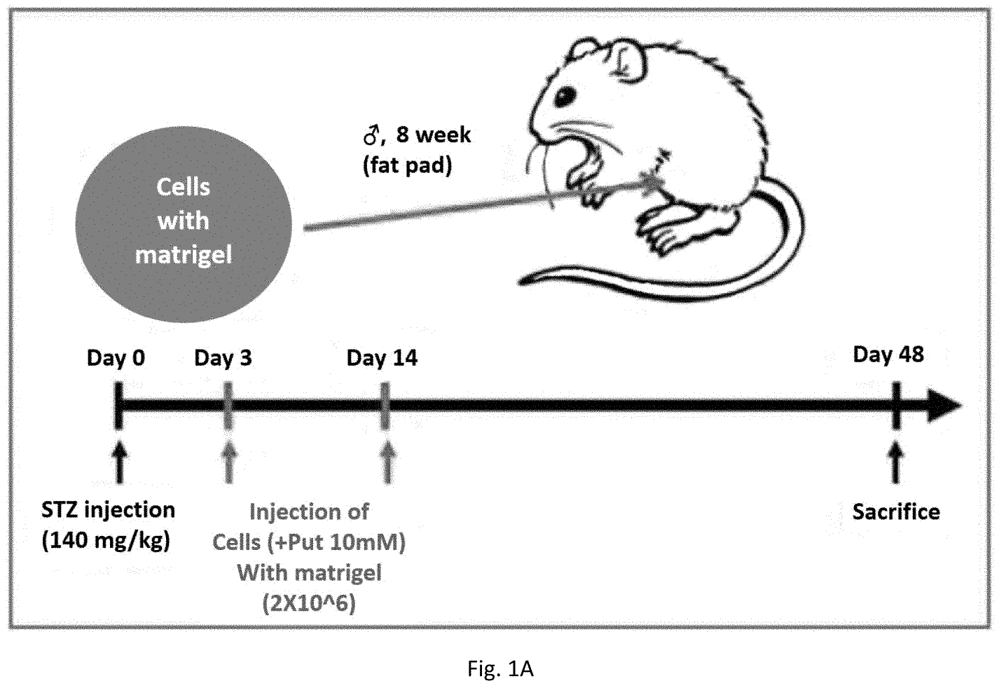

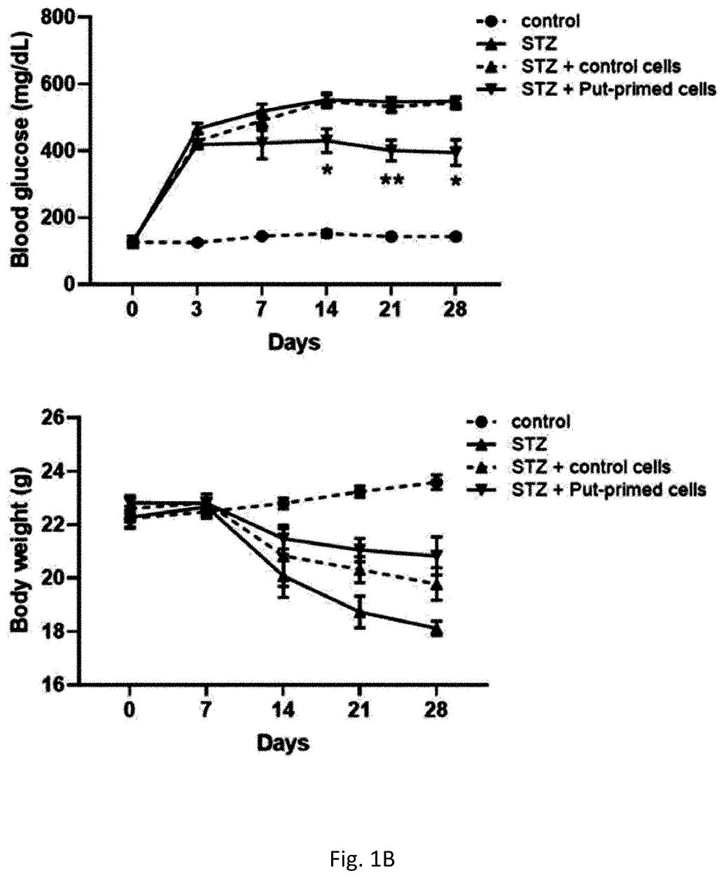

In the diabetes mellitus mouse model, the cells primed with putrescine (2×10⁶ cells) as described above were transplanted into the subcutaneous space twice on days 3 and 14 after STZ administration. Random blood glucose levels and body weight of diabetes mellitus mouse models transplanted with the primed cells were measured for 28 days, which are illustrated in FIG. 1B.

As illustrated in FIG. 1B, random blood glucose levels were increased and body weights were decreased in all of the STZ-administered groups, compared to the control group. However, an increase in blood glucose levels was inhibited and a decrease in body weight caused by diabetes mellitus was suppressed in a group that was transplanted with the cells primed with putrescine (Put-primed cells).

### Example 1.2 Differentiation of Human Bone Marrow Cells into Insulin-Producing Cells

In the present example, it was verified whether human-derived stem cells were induced to differentiate into insulin-producing cells by putrescine. Human bone marrow cells derived from white male donors were purchased from KOMABIOTECH Inc.

Human-derived bone marrow cells (5×10⁶ cells/well) were seeded into 12-well non-coated plates and primed for 6 days in a DMEM culture medium containing 5.5 mM glucose, 10% FBS, and 1% antibiotics, supplemented with putrescine to a concentration of 5 mM. The cells were cultured in suspension on a shaking (30 to 60 rpm) platform in a cell culture incubator (37° C., 5% CO₂).

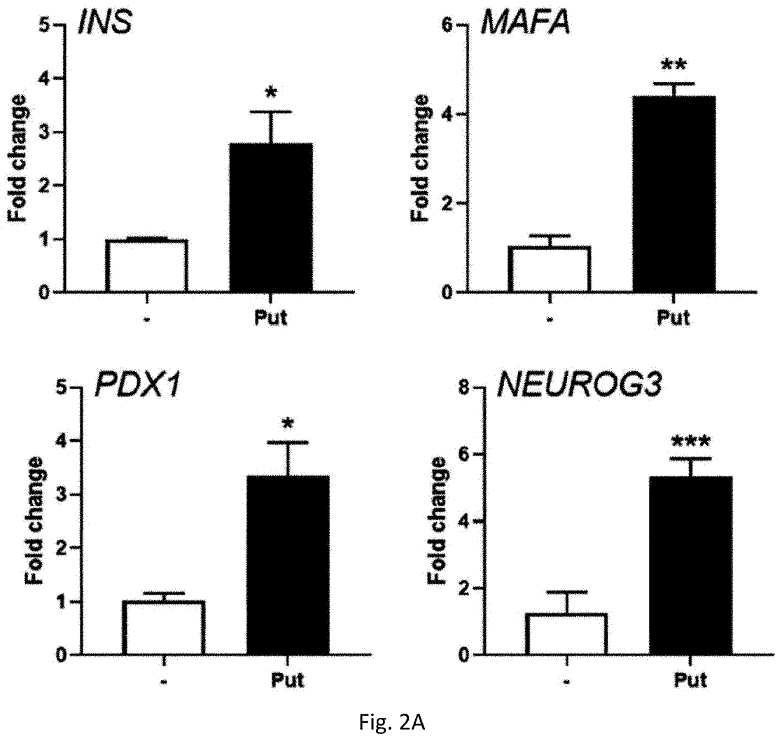

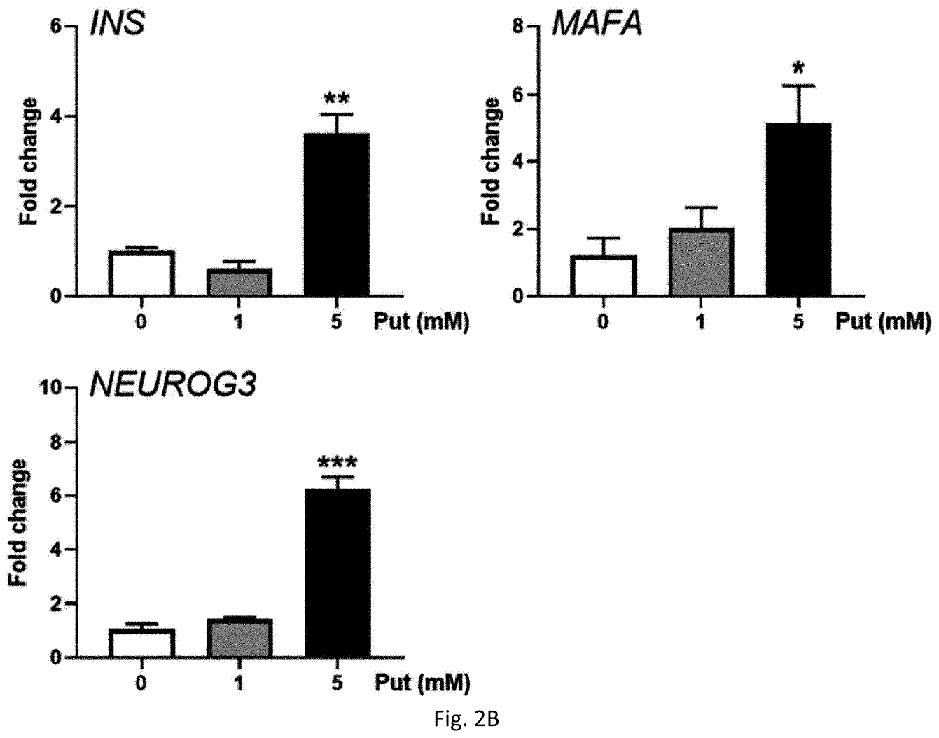

To investigate whether or not insulin-producing cells appeared, the gene expression levels for insulin (INS) and factors (MAFA, PDX-1, and NEUROG3) that promote the differentiation of insulin-producing beta cells were measured using quantitative real-time PCR after harvesting the cells on day 6 after culture. The results are illustrated in FIG. 2A.

Specifically, total RNA was extracted by collecting cells on day 6 after culture. The RNA concentration/quality was evaluated using a NanoDrop spectrophotometer (NanoDrop Technology, Wilmington, Del., USA). The same amount (1 μg) of RNA was reverse-transcribed into cDNA using a reverse transcription kit (Enzynomics, Daejeon, Korea), and quantitative real-time PCR (ABI PRISM 7900, Applied Biosystems) was performed using SYBR Green PCR Master Mix (Applied Biosystems, Foster City, Calif., USA). The gene expressions for insulin (INS), MAFA, PDX-1, NEUROG3 and the like were measured by performing a cycle consisting of 95° C. for 10 minutes, 95° C. for 10 seconds and 60° C. for 30 seconds by repeating the cycle 40 times. The relative mRNA expression of each sample was standardized by GAPDH (control gene) and the statistical analysis was performed using the Student's unpaired t-test. The base sequences of the primers used for amplification are shown in the following Table 1.

TABLE 1

| | | Base sequence | |
|---|---|---|---|
| Primer | | (5′->3′) | SEQ ID NO |
| INS | Forward | CTG CAT CAG AAG | SEQ ID NO: 1 |
| | | AGG CCA TCA AG | |
| | Reverse | GGG TGT GTA GAA | SEQ ID NO: 2 |
| | | GAA GCC TCG | |
| MAFA | Forward | CGC ACG CTC AAG | SEQ ID NO: 3 |
| | | AAC CG | |
| | Reverse | GCC AGC TTC TCG | SEQ ID NO: 4 |
| | | TAT TTC TCC TTGT | |
| PDX-1 | Forward | TTC ACG AGC CAG | SEQ ID NO: 5 |
| | | TAT GAC CTT CAC | |
| | Reverse | GAA GAC AGA CCT | SEQ ID NO: 6 |
| | | GGG ATG CAC A | |
| NEUROG3 | Forward | CTA AGA GCG AGT | SEQ ID NO: 7 |
| | | TGG CAC TG | |
| | Reverse | CCG AGT TGA GGT | SEQ ID NO: 8 |
| | | TGT GCA TT | |
| GAPDH | Forward | CTG CAC CAC CAA | SEQ ID NO: 9 |
| | | CTG CTT AG | |
| | Reverse | AGG CAG GGA TGA | SEQ ID NO: 10 |
| | | TGT TCT GG | |

As illustrated in FIG. 2A, it was confirmed that the mRNA expression of insulin (INS), MAFA, PDX1, and NEUROG3 was significantly increased in human-derived bone marrow cells in which putrescine was added to induce differentiation, compared to a control (indicated as ‘-’ in FIG. 2A) cultured in a culture medium composition which did not include putrescine.

### Example 1.3 Differentiation of Human Umbilical Cord Blood Stem Cells into Insulin-Producing Cells

As another source of human-derived stem cells, umbilical cord blood-derived mononuclear cells (or umbilical cord blood cells herein), which are relatively easily supplied, were treated with putrescine to induce differentiation into insulin-producing cells.

Umbilical cord blood-derived mononuclear cells were freshly isolated using Ficoll-gradient protocol and suspended in a lysis solution (BD Pharmingen) to remove red blood cells.

Differentiation was induced by treating (priming) with putrescine in the same manner as in the method of Example 1.2, except that putrescine was added to a concentration of 1 mM or 5 mM, and the gene expression levels for insulin (INS) and factors (MAFA, NEUROG3) that promote the differentiation into insulin-producing beta cells were measured by performing quantitative real time PCR in the same manner as in the method of Example 1.2. As illustrated in FIG. 2B, the mRNA expression of beta cell-specific genes (INS, MAFA, and NEUROG3) was significantly increased in human-derived umbilical cord blood cells to which 5 mM putrescine was added for differentiation, compared to control cells (indicated as ‘0’ in FIG. 2B) cultured in a culture medium composition which did not include putrescine.

### Example 1.4 Effect on Differentiation into Insulin-Producing Cells by Different Concentrations of Putrescine

### Example 1.4.1 Effect on Differentiation into Insulin-Producing Cells by Different Concentrations of Putrescine

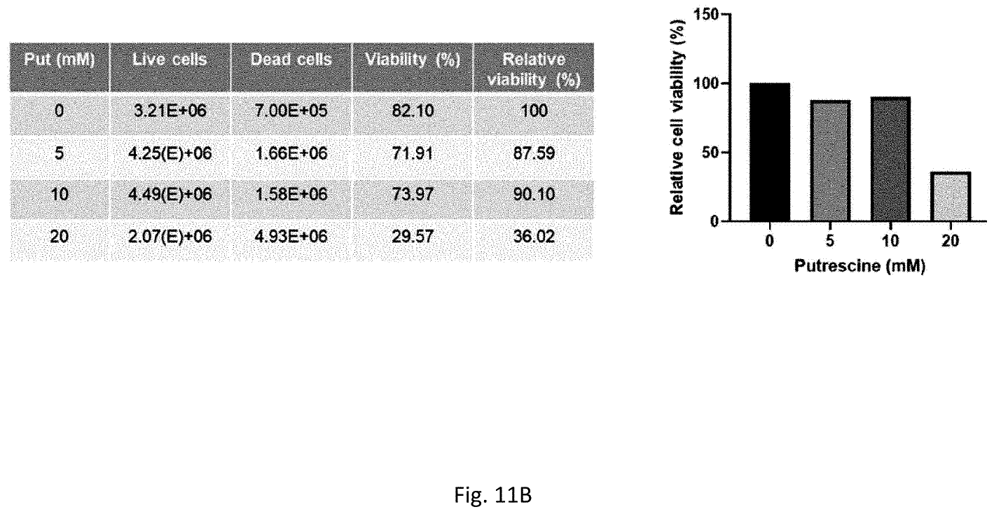

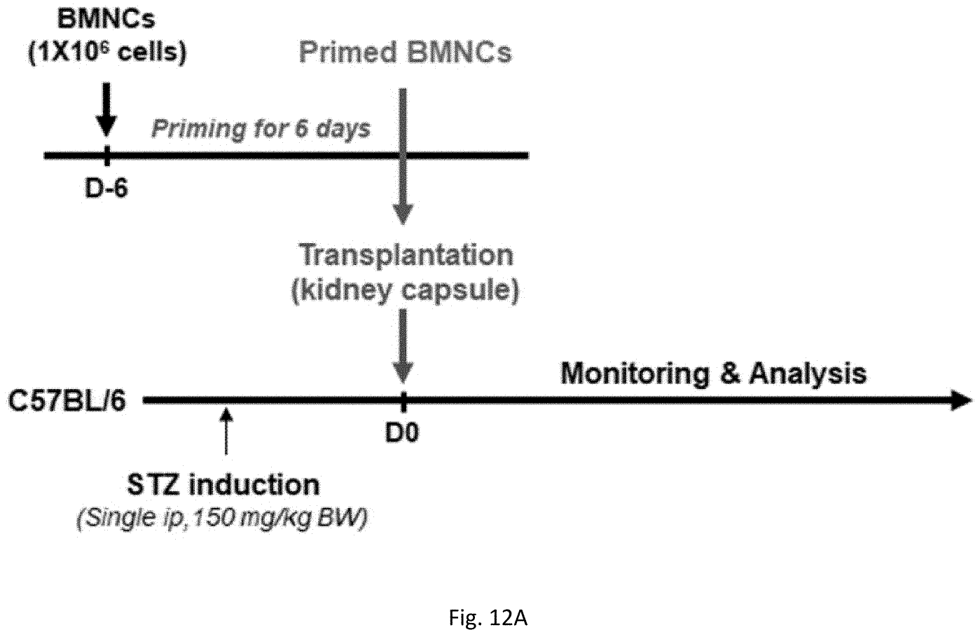

Human umbilical cord blood cells were cultured in a culture medium (putrescine 0 mM) which did not include putrescine or a culture medium including putrescine at various concentrations of 0.1 to 20 mM in the same manner as in Example 1.3. After being cultured for 6 days, cells were collected and subjected to quantitative real-time PCR to measure the gene expression levels for insulin (INS) and factors (MAFA, PDX-1, NEUROG3, or NKX6.1) that promote the differentiation into insulin-producing beta cells, and the gene expression levels are illustrated in FIG. 11A (the primers used to measure the gene expression levels are shown in Tables 1 and 2).

As illustrated in FIG. 11A, the gene expression levels for insulin and the factors that promote the beta cell differentiation were significantly increased in the cells primed with putrescine at a concentration of 5 mM or more.

### Example 1.4.2 Analysis of Cell Viability by Different Concentrations of Putrescine

Human umbilical cord blood cells were cultured in a culture medium (putrescine 0 mM) which did not include putrescine or a culture medium including putrescine at a concentration of 5 to 20 mM in the same manner as in Example 1.3. After being cultured for 6 days, cells were collected, and live and dead cells were stained with acridine orange (AO, live cell stain) and propidium iodide (PI, dead cell stain), respectively, and then the cell number and the cell viability (%) were acquired using a Cellometer Fluorescent Viability Cell Counter K2, as shown in FIG. 11B.

As illustrated in FIG. 11B, the number of cells in the culture medium composition containing 20 mM putrescine was sharply decreased. Based on this finding, putrescine concentrations above 20 mM appear to affect the cell viability.

### Example 2. Differentiation into Insulin-Producing Cells by Co-Treatment of Putrescine and Glucosamine

Mouse bone marrow cells were isolated from 8-week-old male C57BL/6 mice (Orientbio Inc.) in the same manner as in Example 1.1. Bone marrow cells in DMEM culture medium (Dulbecco's Modified Eagle's Medium) (standard medium) containing 5.5 mM glucose, 10% FBS, and 1% antibiotics were seeded at 5×10⁶ cells/ml into a 12-well non-coated plate, treated with glucosamine (GlcN) and putrescine (Put) to a concentration of 10 mM each or together, and cultured for 6 days in suspension for differentiation.

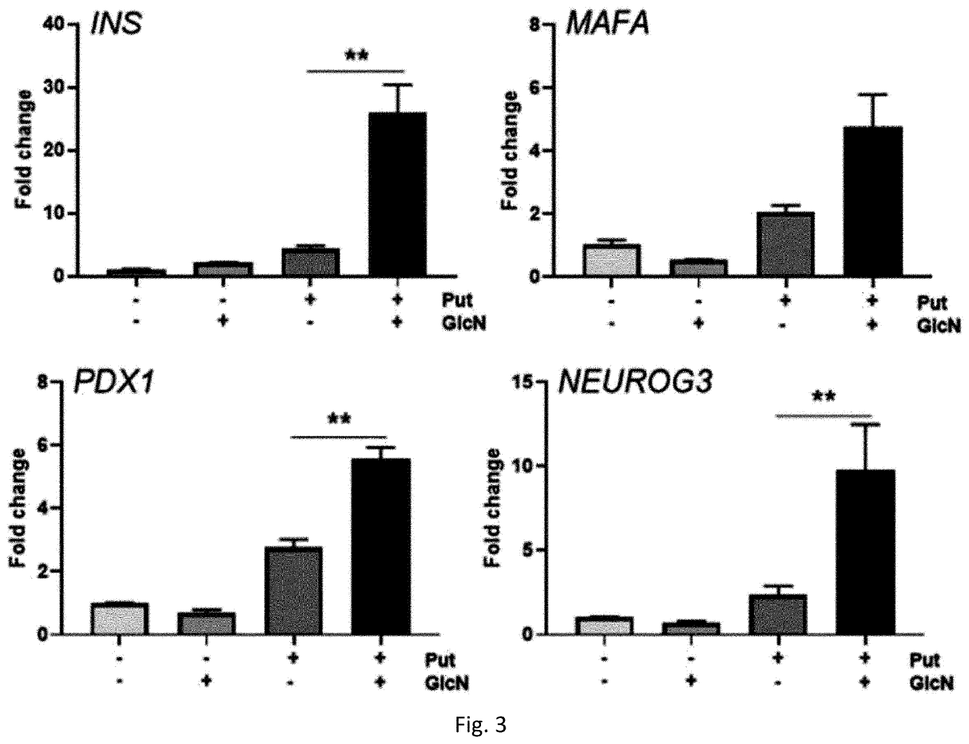

To investigate whether or not insulin-producing cells appeared, the gene expression levels of insulin (INS), MAFA, PDX-1, and NEUROG3 were measured by a quantitative real-time PCR method, as in the method of Example 1.2, which are shown in FIG. 3.

As illustrated in FIG. 3, when cells were treated with glucosamine or putrescine compared to the control, the gene expression of insulin and factors that promote the differentiation into insulin-producing cells was changed, and the mRNA expression of insulin (INS), MAFA, PDX-1 and NEUROG3 was all synergistically increased in the group co-treated with putrescine and glucosamine compared to the group treated with putrescine or glucosamine alone. Particularly, the insulin expression was synergistically increased when differentiation was induced by the co-treatment with glucosamine and putrescine (26-fold compared to the control), rather than when differentiation was induced by the treatment with glucosamine (2-fold compared to the control) or putrescine (4.3-fold compared to the control) alone.

### Example 3. Optimization for Differentiation into Insulin-Producing Cells

In the present example, it was intended to investigate the effect of a composition containing putrescine, glucosamine, and nicotinamide on differentiation into insulin-producing cells.

Samples were divided into (1) putrescine alone-treated group (5 mM), (2) putrescine (5 mM) and glucosamine (5 mM)-co-treated group, (3) putrescine (5 mM), glucosamine (5 mM), and nicotinamide (10 mM)-co-treated group, and (4) control (non-treated group). Umbilical cord blood cells (prepared in the same manner as in Example 1.3) and mouse bone marrow cells (prepared in the same manner as in Example 1.1) were cultured in suspension to induce the differentiation into insulin-producing cells.

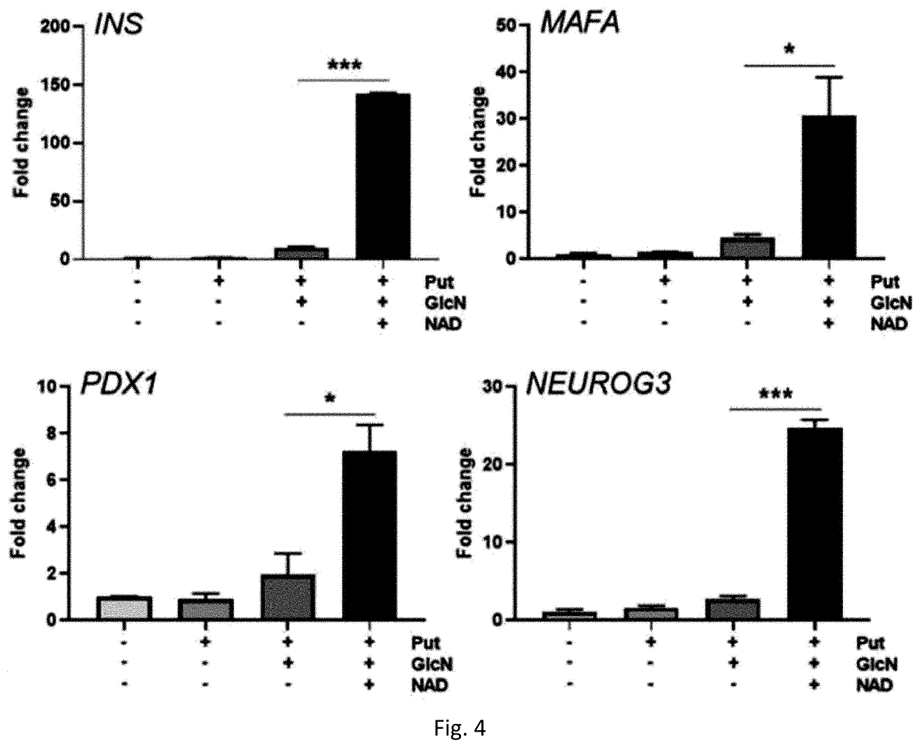

On day 6 after priming the umbilical cord blood cells or mouse bone marrow cells for differentiation, total RNA was extracted to perform quantitative real-time PCR, similarly to the method of Example 1.2, and the mRNA expression of insulin and beta cell differentiation-related genes (MAFA, PDX1, or NEUROG3) was determined, as shown in FIGS. 4 (in primed umbilical cord blood cells) and 5 (in primed mouse bone marrow cells).

As illustrated in FIG. 4, the mRNA expression of insulin and beta cell differentiation-related genes was synergistically increased in the co-treated group of putrescine, glucosamine, and nicotinamide, compared to the co-treated group of putrescine and glucosamine. In the co-treated group of putrescine, glucosamine, and nicotinamide, mRNA expression of insulin was increased about 142.5-fold compared to the control, and about 14-fold or more, compared to the co-treated group of glucosamine and putrescine (the expression level of insulin was increased 10-fold compared to the control).

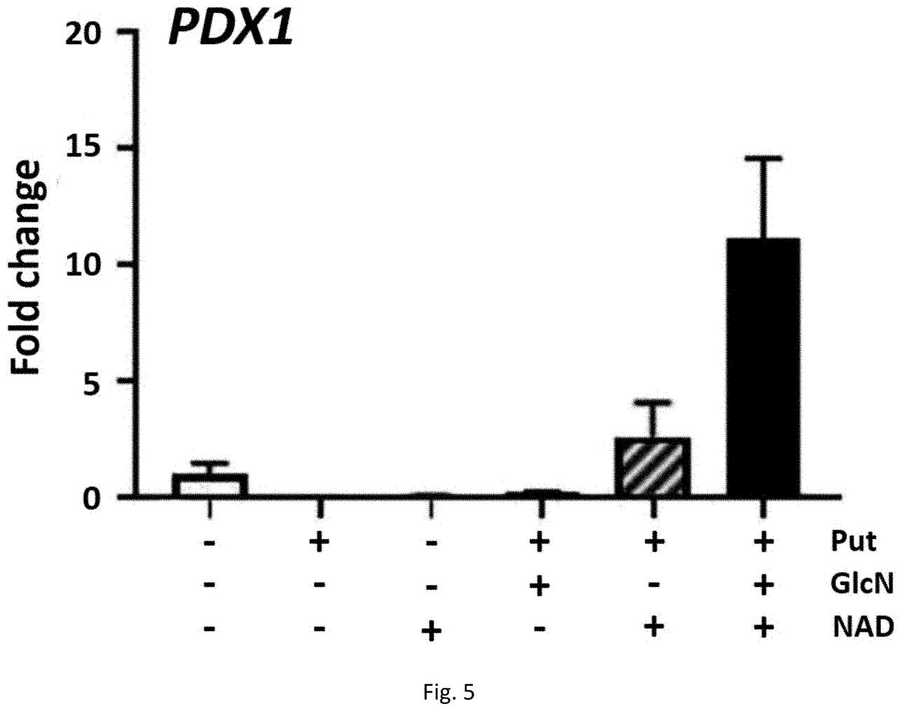

As illustrated in FIG. 5, the expression of a beta cell differentiation-related gene (PDX1) was synergistically significantly increased in the group, in which mouse bone marrow cells were co-treated with putrescine, glucosamine, and nicotinamide, compared to each group in which differentiation was induced by treating mouse bone marrow cells individually with putrescine, glucosamine, or nicotinamide.

### Example 4. Efficacy of Inducing Differentiation into Insulin-Producing Cells by Different Cell Culture Media

Umbilical cord blood cells were isolated in the same manner as in Example 1.3, and the differentiation effect on culture medium was determined. Umbilical cord blood mononuclear cells were cultured for 6 days in suspension under conditions similar to those in Example 1.1, in a standard culture medium (DMEM) or CMRL1066 culture medium, supplemented with glucosamine, putrescine, and nicotinamide, such that the concentrations of glucosamine, putrescine, and nicotinamide were 5 mM, 5 mM, and 10 mM, respectively. On day 6 after the culture, the expression levels of genes were compared in the cells in which differentiation was induced in the DMEM or CMRL1066 culture medium, respectively, using RT-PCR and quantitative real-time PCR. The sequences of primers used for RT-PCR and real-time PCR are shown in Tables 1 and 2. The conditions for quantitative real-time PCR were the same as in Example 1.2, and RT-PCR was performed with the following steps; 1) initial denaturation of 94° C. for 5 minutes; 2) amplification of 94° C. for 30 seconds, 60° C. for 30 seconds, and 72° C. for 1 minute by repeating the cycle 30 times; 3) final extension of 72° C. for 10 minutes.

TABLE 2

| | | Base sequence | |
|---|---|---|---|
| Primer | | (5′->3′) | SEQ ID NO |
| NEUROD1 | Forward | TAA GAC GCA GAA | SEQ ID NO: 11 |
| | | GCT GTC CA | |
| | Reverse | GTC CGA GGA TTG | SEQ ID NO: 12 |
| | | AGT TGC AG | |
| NKX6.1 | Forward | GAA CCG CCG GAC | SEQ ID NO: 13 |
| | | CAA GT | |
| | Reverse | GTC GTC CGA GTT | SEQ ID NO: 14 |
| | | GGG ATC CAG | |
| GAPDH | Forward | CTG CAC CAC CAA | SEQ ID NO: 9 |
| | | CTG CTT AG | |
| | Reverse | AGG CAG GGA TGA | SEQ ID NO: 10 |
| | | TGT TCT GG | |

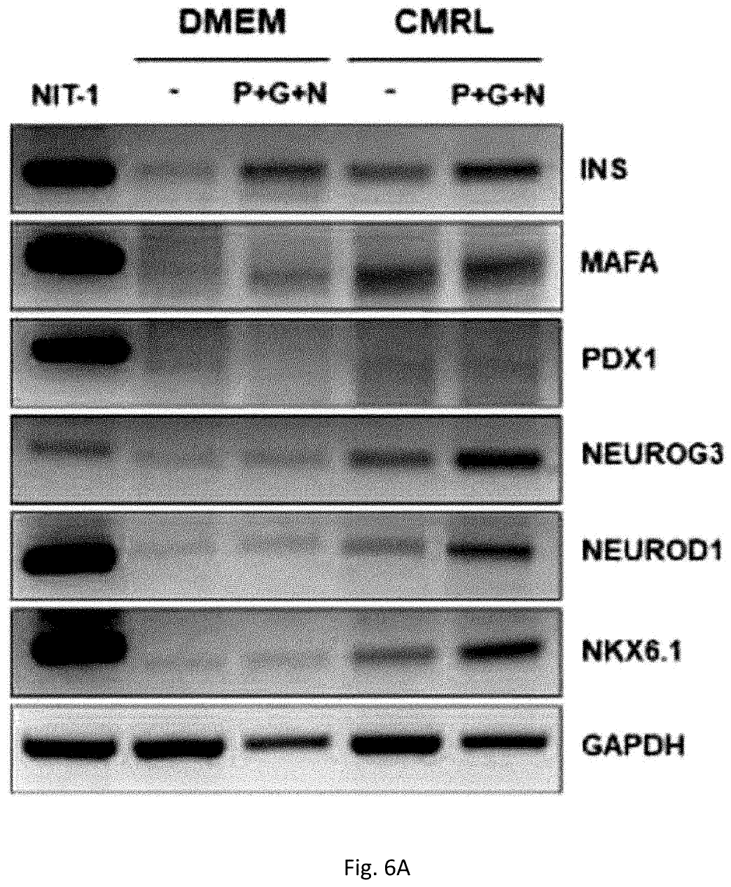

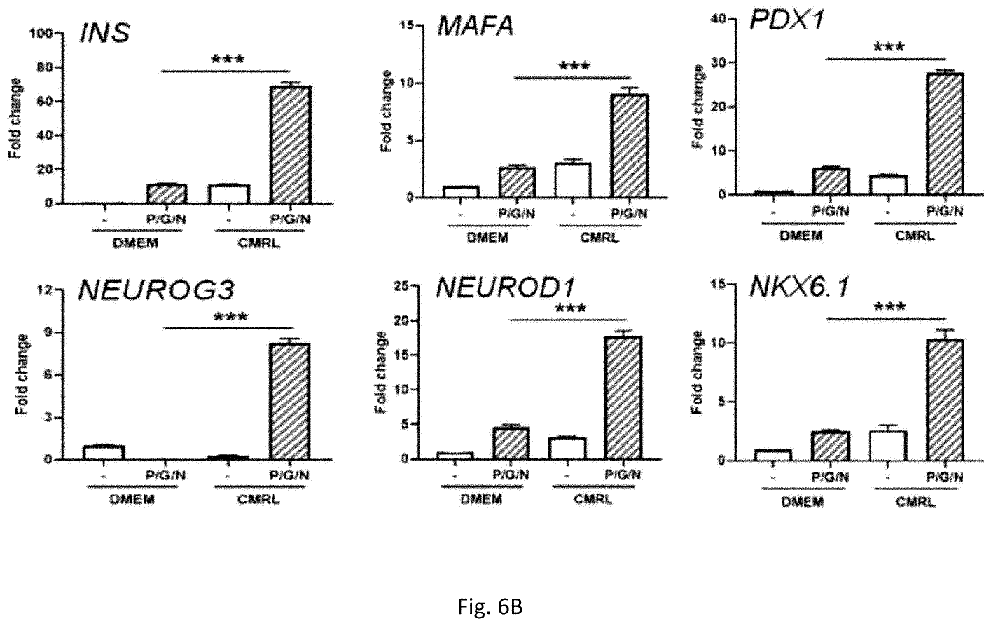

FIG. 6A illustrates the results of RT-PCR, and FIG. 6B illustrates the results of quantitative real-time PCR. As shown in FIGS. 6A and 6B, the mRNA expression of insulin and beta cell differentiation-related genes was significantly increased when cells were cultured in a CMRL 1066 culture medium to which glucosamine, putrescine, and nicotinamide were added.

### Example 5. Verification of Ability to Differentiate into Insulin-Producing Cells Using Human Embryonic Stem Cell-Derived Mesenchymal Stem Cells (E-MSCs)

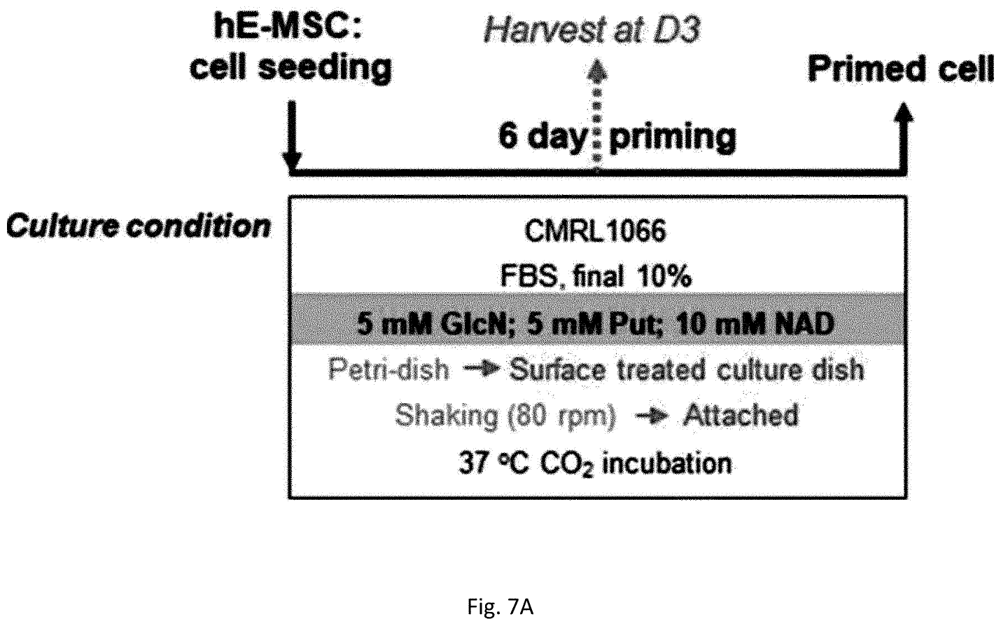

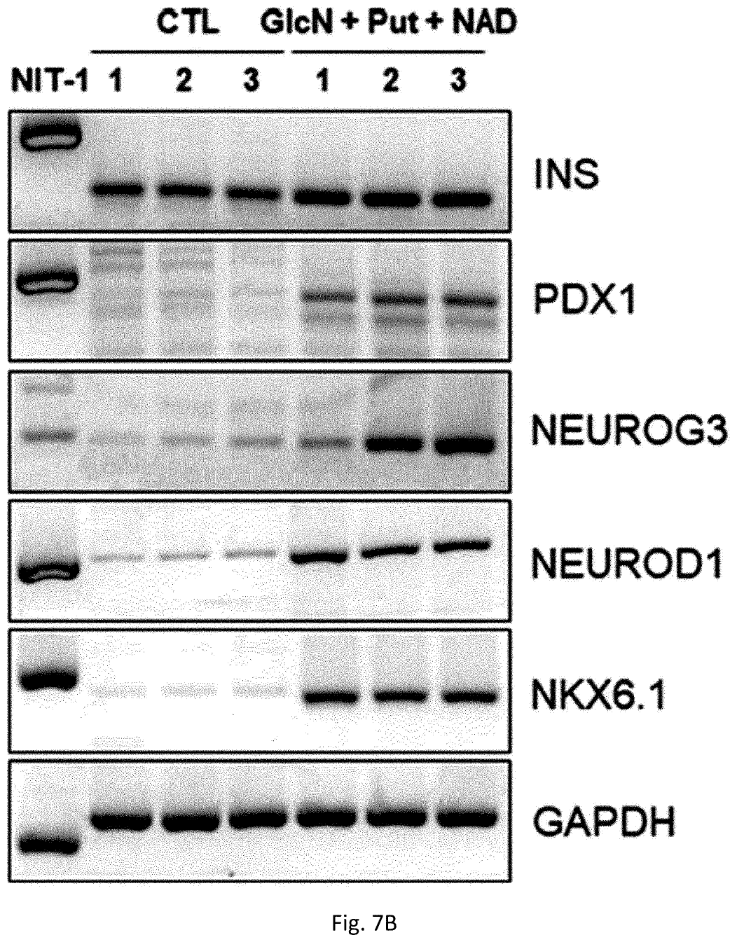

Human embryonic stem cell-derived mesenchymal stem cells (E-MSCs) (provided by Seoul National University Hospital) were cultured adherently in a CMRL 1066 culture medium containing 10% FBS and 1% antibiotics in a surface treated culture dish under the conditions of 37° C. and 5% CO₂, and primed with glucosamine (5 mM), putrescine (5 mM), and nicotinamide (10 mM). Cells were collected on day 3 after priming, and total RNA was extracted to perform RT-PCR on the gene expression for insulin and beta cell differentiation-related genes (PDX1, NEUROG3, NEUROD1, or NKX6.1), similarly to the method of Example 4, and the results are shown in FIG. 7B. In FIG. 7B, NIT-1 is an insulin-producing cell line (beta cell line; insulinoma), in which differentiation was completed, and CTL means a control which was cultured in a CMRL 1066 culture medium in the absence of glucosamine, putrescine, or nicotinamide.

As illustrated in FIG. 7B, it was observed that when differentiation was induced by treating human E-MSCs with a combination of glucosamine, putrescine, and nicotinamide, the mRNA expression of insulin and beta cell differentiation-related genes was significantly increased just on day 3 after the treatment. Therefore, it was confirmed that the method for inducing differentiation into insulin-producing cells (composition for inducing differentiation) could be applied not only to adult stem cells, but also to embryonic stem cell-derived mesenchymal stem cells (embryonic stem cells).

### Example 6. Efficacy of Differentiation into Insulin-Producing Cells Relative to the Treatment Time of a STAT3 Inhibitor

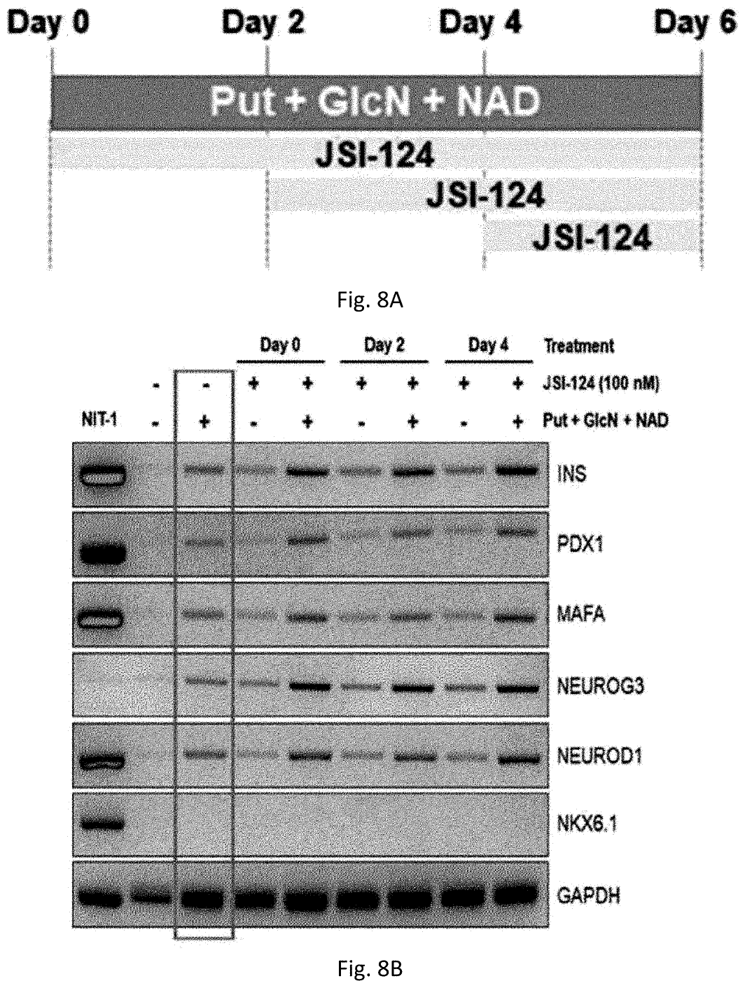

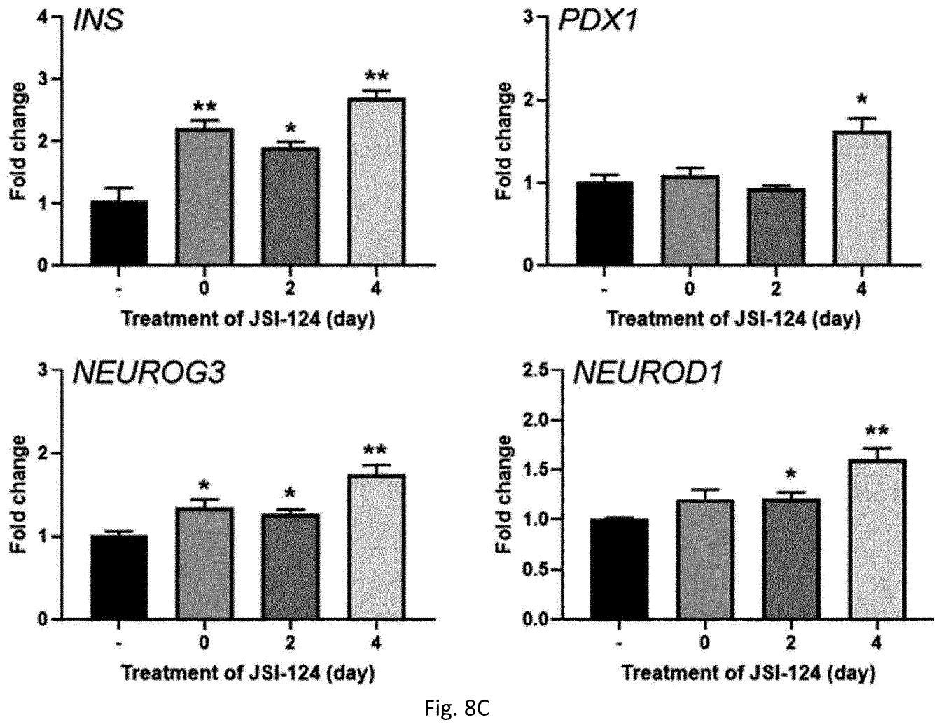

After mouse bone marrow cells were cultured under conditions similar to those in Example 1.1 by treating glucosamine, putrescine, and nicotinamide together in a CMRL 1066 culture medium, the cells were further treated with a STAT3 inhibitor JSI-124 (Calbiochem, DongNam Chemical) on day 0, 2, or 4 after the culture, such that the concentration of JSI-124 in the culture medium was 100 nM (0.1 μM), and then the cells were collected on day 6. In order to confirm the appearance of insulin-producing cells, total RNA was extracted from the collected cells similarly to the method in Example 1.2, and the expression levels of insulin (INS) and beta cell differentiation-related genes (PDX1, NEUROG3, or NEUROD1) were confirmed by RT-PCR and real-time PCR, which are illustrated in FIGS. 8B and 8C. FIGS. 8B and 8C show RT-PCR and real-time PCR results, respectively, and in FIG. 8C, ‘−’ means a group which is treated with only putrescine, glucosamine, and nicotinamide, excluding a STAT3 inhibitor.

As illustrated in FIGS. 8B and 8C, it could be confirmed that in the experimental group to which the STAT3 inhibitor was added, the expression levels of genes promoting differentiation into insulin-producing cells were increased, and in particular, when JSI-124 was added on day 4 after the culture, the mRNA expression levels of insulin (INS) and beta cell differentiation-related genes (PDX1, NEUROG3, or NEUROD1) were significantly increased compared to those when treated with JSI-124 at other time points.

### Example 7. Efficacy of Differentiation into Insulin-Producing Cells by Different Types of STAT3 Inhibitors

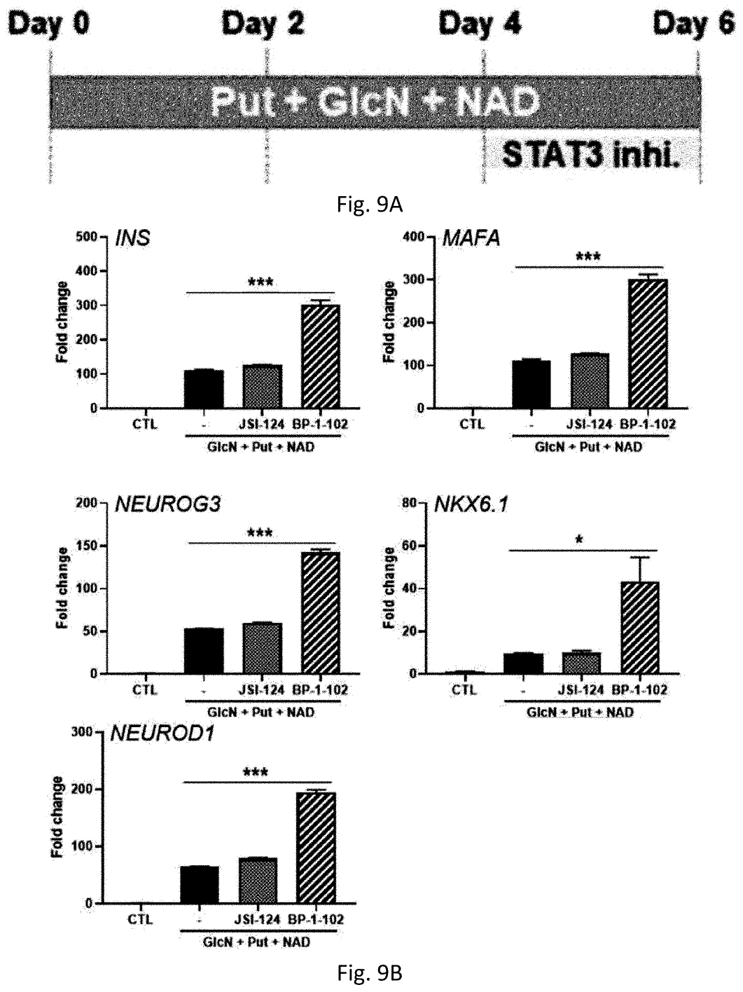

In order to investigate the differentiation effect depending on the type of STAT3 inhibitor, human umbilical cord blood cells isolated in the same manner as in Example 1.3 were cultured in a CMRL1066 culture medium to which glucosamine, putrescine, and nicotinamide (5 mM putrescine, 5 mM glucosamine, and 10 mM nicotinamide) were added together, and further treated with various types of STAT3 inhibitors (JSI-124 (0.1 μM; Calbiochem, DongNam Chemical), CPT (1 μM; Sigma, DongNam Chemical), or BP-1-102 (10 μM; Calbiochem, DongNam Chemical)) at a suitable concentration at which each STAT3 has an inhibitory activity. Cells were collected on day 6 after the culture to confirm the appearance of insulin-producing cells. Total RNA was extracted from the collected cells similarly to the method in Example 1.2, and the mRNA expression levels of insulin (INS) and beta cell differentiation-related genes (NEUROG3, NEUROD1, or NKX6.1) were confirmed by RT-PCR and real-time PCR, which are illustrated in FIG. 9B.

It could be seen that among the STAT3 inhibitors, the mRNA expression levels of insulin and beta cell differentiation-related genes were significantly increased in the order of CPT <JSI-124<BP-1-102, when each inhibitor was additionally treated to cells supplemented with the composition comprising glucosamine, putrescine, and nicotinamide (the results for CPT are not illustrated in FIG. 9B). In particular, it was confirmed that when cells were further treated with BP-1-102 on day 4, the mRNA expression of insulin was increased about 3-fold or more compared to when cells were treated only with the three factors of glucosamine, putrescine, and nicotinamide.

### Example 8. Assessment of Insulin Secretion Capability by Treatment with a STAT3 Inhibitor

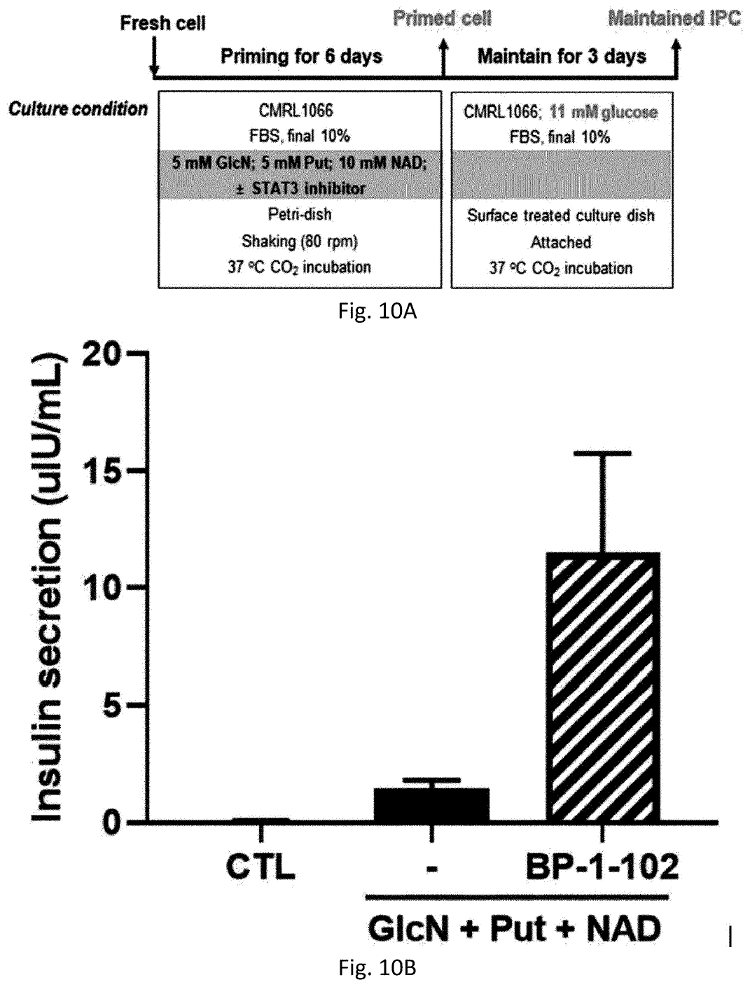

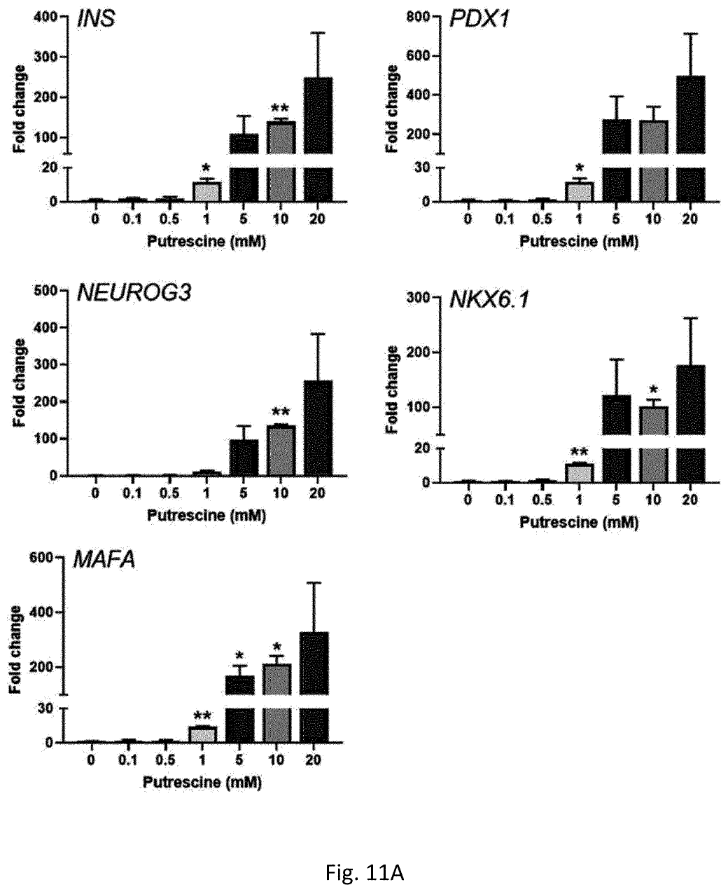

As in Example 7, umbilical cord blood cells were primed in the CMRL1066 culture medium containing glucosamine, putrescine, and nicotinamide in suspension, and BP-1-102 was further added on day 4 to facilitate the differentiation into insulin-producing cells. The primed cells were collected on day 6, washed with PBS to remove the remaining differentiation-inducing factors (glucosamine, putrescine, nicotinamide, and BP-1-102), re-suspended in a CMRL1066 culture medium containing 11 mM glucose, along with 10% FBS and 1% antibiotics and then seeded onto a 6-well surface treated culture plate. After 3 days, the supernatants were collected, and insulin levels were measured using an insulin ELISA kit (Ultrasensitive Insulin ELISA, Catalog #80-INSHUU-E01.1; ALPCO, Genomicsone Co., Ltd.), as described in FIG. 10A.

As shown in FIG. 10B, it was confirmed that umbilical cord blood cells differentiated into insulin-producing cells secreted insulin extracellularly. It was also confirmed that when differentiation was induced by treatment together with a STAT3 inhibitor BP-1-102 on day 4 after the culture, the insulin secretion was increased 10-fold or more compared to when only three differentiation-inducing factors (glucosamine, putrescine, and nicotinamide) were added.

### Example 9. Safety and Validation of Efficacy of Transplantation of Differentiated Mouse Bone Marrow Cells in Diabetes Mellitus Mouse Models

In the present example, it was intended to investigate whether mouse bone marrow cells primed with a composition comprising putrescine, glucosamine, nicotinamide and a STAT3 inhibitor had the capability to regulate blood glucose levels in diabetes mellitus mouse models.

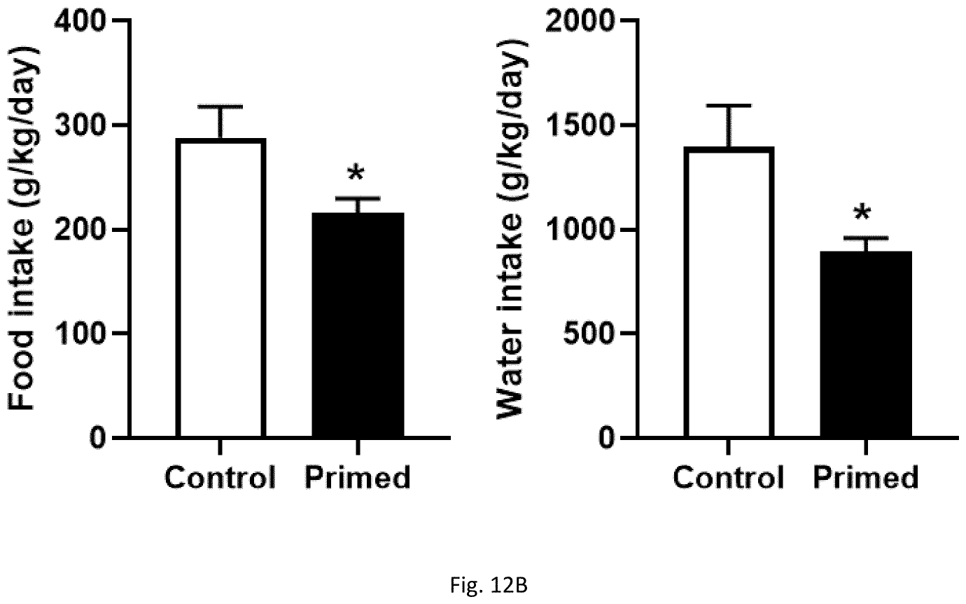

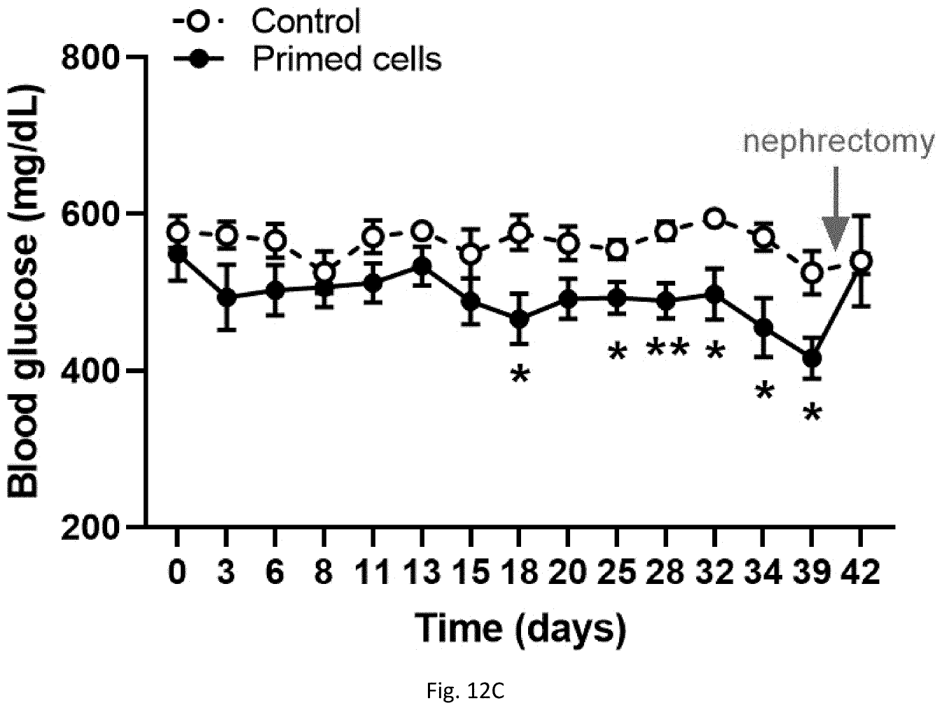

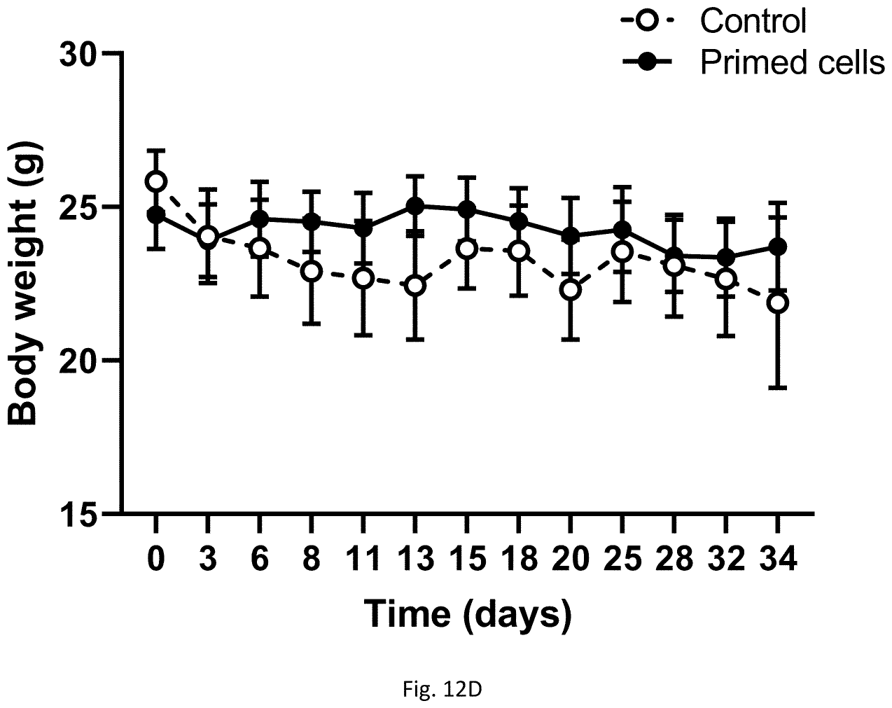

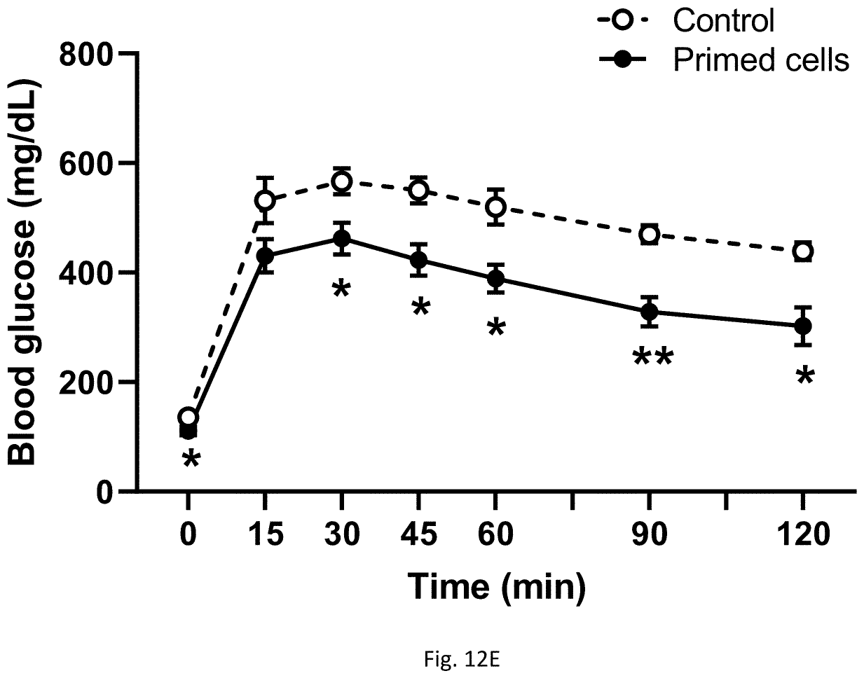

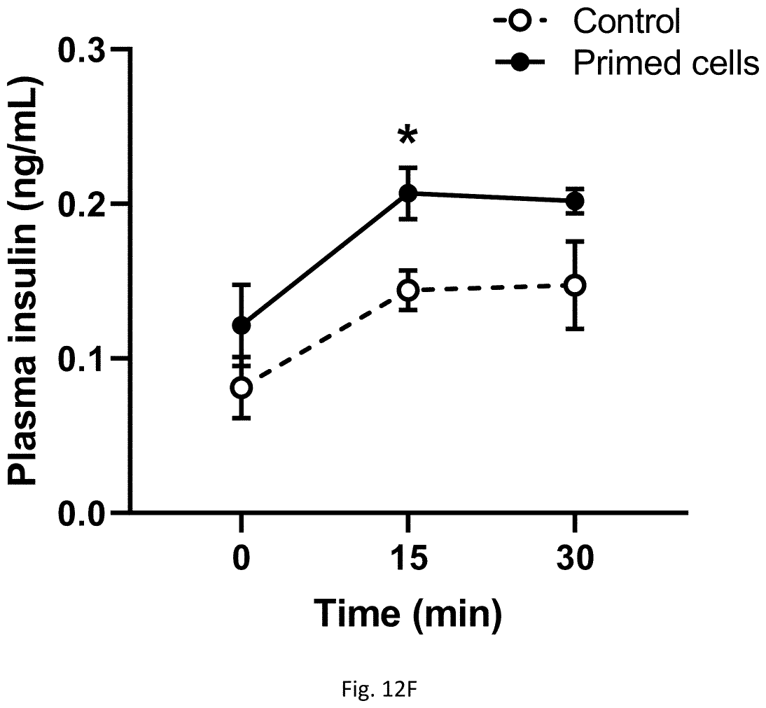

Mouse bone marrow cells (referred to BMNCs herein in FIG. 12A) were isolated from 8-week-old male C57BL/6 mice (Seoul National University Institute of Laboratory Animal Resources), in the same manner as described in Example 1.1. BMNCs were seeded into a 12-well non-coated plate at 5×10⁶ cells/ml in a CMRL1066 medium containing 10% FBS and 1% antibiotics, and primed for 6 days with putrescine, glucosamine, nicotinamide and a STAT3 inhibitor. Primed or non-primed BMNCs (1×10⁶ cells/mouse) were resuspended in the same volume of Matrigel (Corning Life Sciences) for kidney capsule transplantation. Diabetes mellitus was induced in male C57BL/6 mice aged 8 weeks (Seoul National University Institute of Laboratory Animal Resources) by a single intraperitoneal injection of 150 mg/kg of streptozotocin (STZ, Sigma). Three-day post STZ stimulation, animals with random blood glucose levels 400 mg/dL for three consecutive days were considered as STZ-induced diabetes mellitus mice. As illustrated in FIG. 12A, the primed cell-containing Matrigel solution was transplanted into a kidney capsule after the kidney was exposed through a small lumbar incision on day 7 after STZ administration. Random blood glucose levels, body weight, and food and water intake were measured every 3-4 day for 42 days. As shown in FIGS. 12B to 12D, the STZ-treated control mice implanted with non-primed BMNCs exhibited metabolic parameters including a significant increase in both food and water intake, blood glucose levels and persistent weight loss. However, the primed cell-implanted mice showed decreased blood glucose levels as early as day 18 following transplantation. Abnormally increased food and water intake decreased after transplantation. The mice also maintained their body weight. Diabetes mellitus mouse models transplanted with the primed BMNCs showed significantly lower fasting blood glucose levels and improved glucose tolerance following intraperitoneal glucose challenge at a concentration of 1 g/kg body weight on day 27 after grafting, compared to the matched controls, as illustrated in FIG. 12E.

To confirm the capability of insulin secretion in vivo following glucose loading, mice were fasted in the same manner as above on day 34 after transplantation, and blood samples were obtained via tail vein at baseline insulin levels (0 min) before and 15, 30 minutes after a bolus of glucose at 2 g/kg body weight. Plasma insulin levels were measured using the Mouse Ultrasensitive Insulin ELISA (ALPCO). In vivo glucose-stimulated insulin secretion from the primed cell-transplanted mice displayed significantly increased levels of plasma insulin at 15 min post-glucose injection compared to the corresponding controls in STZ-induced mice, as illustrated in FIG. 12F. These results suggested that BMNC-derived insulin-producing cells are functional in vivo and capable of lowering hyperglycemia in diabetes mellitus mouse models.

### Example 10. The Presence of Insulin- and PDX1-Expressing Cells in Primed Cell-Transplanted Kidney

In order to confirm whether the primed cells transplanted under the kidney capsule were involved in the amelioration of hyperglycemia, the kidney sections from the nephrectomized mice were subjected to immunofluorescence staining using anti-insulin and anti-PDX1 antibodies.

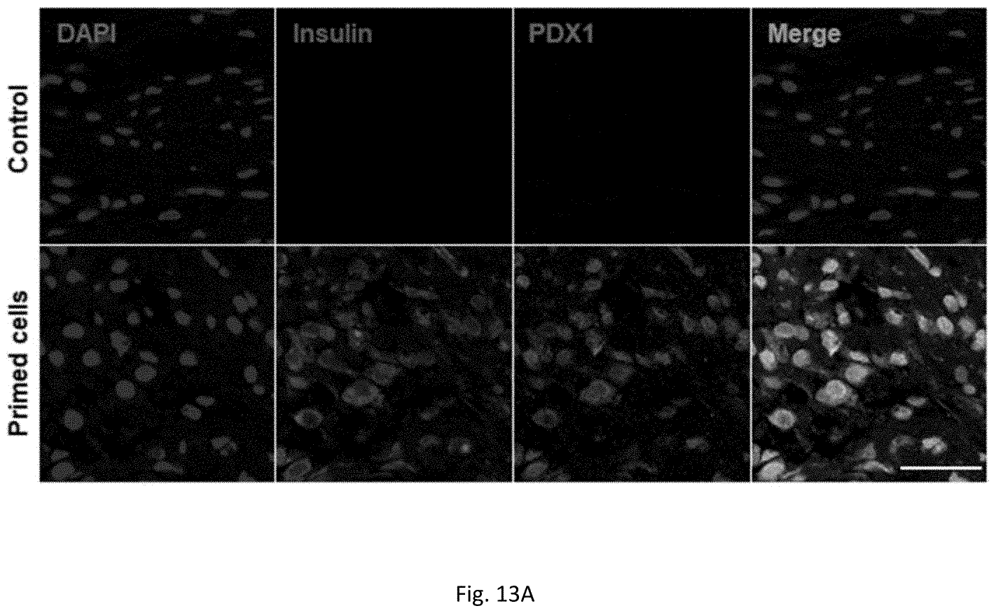

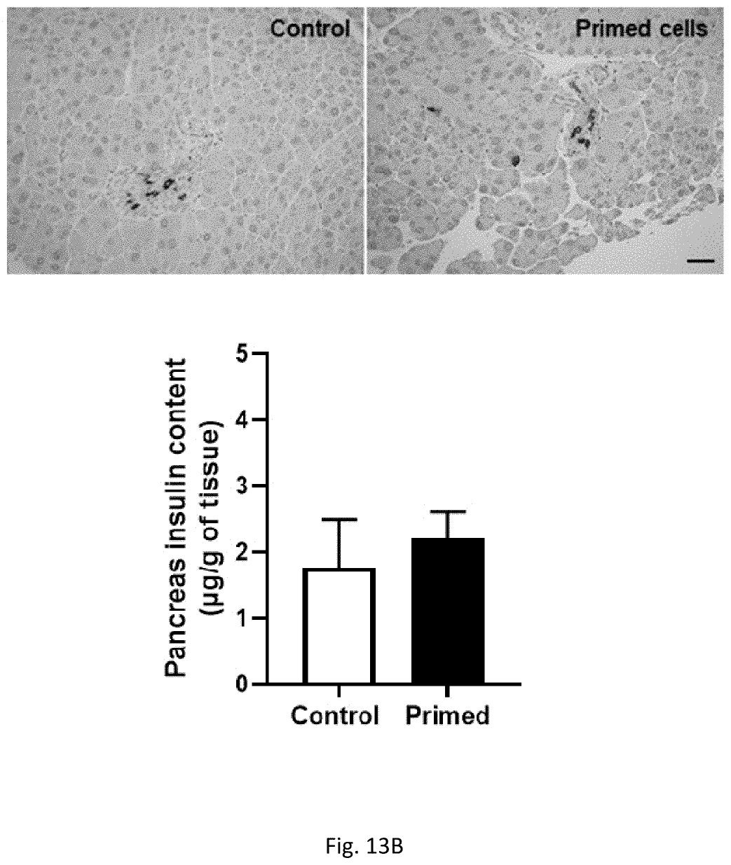

As illustrated in FIG. 13A, immunofluorescence staining confirmed the presence of insulin- and PDX1-expressing cells in the primed cell-transplanted kidney; however, the expression of insulin and PDX1 was not observed in the non-primed cell-transplanted kidney. In FIG. 13B, to rule out the possibility of endogenous pancreatic beta cell regeneration, immunohistochemistry with anti-insulin antibody from the pancreatic tissues was performed on day 42 from the STZ-induced mice transplanted with primed cells.

As illustrated in FIG. 13B, the pancreas from the mice grafted with primed cells showed near complete loss of pancreatic islets, which is comparable to the diabetic controls, and no evidence of endogenous beta cell regeneration. As an additional assessment, the pancreatic insulin content in these mice was quantitatively determined. Pancreatic tissues cut in half were placed into acid-ethanol buffer containing 1.5% HCl in 70% EtOH solution overnight at −20° C. for insulin extraction. The tissues were then homogenized and centrifuged, and the supernatants were neutralized with 1 M Tris with a pH of 7.5 to measure the insulin content by the Mouse Insulin ELISA kit (ALPCO). Similar to the results of immunohistochemistry, no significant difference between the groups (control vs. primed cells) was observed. Thus, the primed cells respond to glucose challenge in vivo by releasing insulin.

### INDUSTRIAL APPLICABILITY

The composition for inducing differentiation into insulin-producing cells in the present invention can produce insulin-producing cells using various types of stem cells in a short period of time by a simple method, and the insulin-producing cells differentiated by the method of the present invention or insulin produced therefrom can be effectively used for the treatment of diabetes mellitus. In the current situation, where there is no clear treatment method for diabetes mellitus other than insulin injection therapy, the present invention is expected to be an innovative technology for the treatment of diabetes mellitus, in that insulin-producing cells can be easily produced by priming the various types of adult or embryonic stem cells with the composition comprising putrescine, glucosamine, nicotinamide and a STAT3 inhibitor for a short period of time. Further, the composition provided in the present invention can be free from debates on stability and ethics, minimizes an in vitro manipulation stage, and easily mass-produce insulin-producing cells in a short period of time without using an expensive differentiation-inducing factor, gene manipulation or the like, and thus the composition is expected to be commercialized and applicable in various fields.

1. A composition for inducing differentiation into insulin-producing cells, comprising putrescine, or one or more selected from the group consisting of putrescine, glucosamine, and nicotinamide.

2. The composition of claim 1, wherein the composition further comprises a STAT3 inhibitor.

3. The composition of claim 2, wherein the STAT3 inhibitor is one or more selected from the group consisting of JSI-124, BP-1-102, and CPT.

4. The composition of claim 1, wherein the putrescine is comprised at a concentration of 1 to 20 mM.

5. The composition of claim 1, wherein the glucosamine is comprised at a concentration of 1 to 20 mM.

6. The composition of claim 1, wherein the nicotinamide is comprised at a concentration of 5 to 20 mM.

7. The composition of claim 2, wherein the STAT3 inhibitor is comprised at a concentration of 0.1 to 50 μM.

8. The composition of claim 1, wherein the composition is capable of inducing differentiation of adult stem cells, embryonic stem cells, and a combination thereof into insulin-producing cells.

9. The composition of any one of claims 1 to 8, wherein the composition further comprises one or more culture media selected from the group consisting of CMRL1066, a Dulbecco's Modified Eagle's Medium (DMEM), a minimal essential medium (MEM), Basal Medium Eagle (BME), RPMI1640, F-10, F-12, an α minimal essential medium (αMEM), a Glasgow's minimal essential medium (GMEM), McCoy's 5A, a 199 medium, and endothelial growth medium MV2.

10. A culture medium composition for inducing differentiation into insulin-producing cells, comprising the composition for inducing differentiation into insulin-producing cells according to any one of claims 1 to 8, and a culture medium.

11. The culture medium composition of claim 10, wherein the culture medium is one or more culture media selected from the group consisting of CMRL1066, a Dulbecco's Modified Eagle's Medium (DMEM), a minimal essential medium (MEM), Basal Medium Eagle (BME), RPMI1640, F10, F-12, an α minimal essential medium (αMEM), a Glasgow's minimal essential medium (GMEM), McCoy's 5A, a 199 medium, and endothelial growth medium MV2.

12. A method for inducing differentiation into insulin-producing cells, the method comprising culturing isolated cells in the culture medium composition according to claim 10.

13. The method of claim 12, wherein the culturing step comprises culturing isolated cells under conditions of a temperature of 35 to 38° C. and 5% CO₂ in the culture medium composition for inducing differentiation for 3 days to 6 days.

14. The method of claim 12, wherein the culture medium further comprises one or more culture media selected from the group consisting of CMRL1066, a Dulbecco's Modified Eagle's Medium (DMEM), a minimal essential medium (MEM), Basal Medium Eagle (BME), RPMI1640, F10, F-12, an α minimal essential medium (αMEM), a Glasgow's minimal essential medium (GMEM), McCoy's 5A, a 199 medium, and endothelial growth medium MV2.

15. The method of claim 12, wherein the method comprises culturing cells by adding a STAT3 inhibitor to the culture medium composition on day 3 to day 5 after cell culture.

16. The method of claim 12, wherein the STAT3 inhibitor is one or more selected from the group consisting of JSI-124, BP-1-102, and CPT.

17. The method of claim 12, wherein the isolated cells are adult stem cells, embryonic stem cells, or a combination thereof.

18. A method for preventing or treating diabetes mellitus, the method comprising administering a composition comprising the composition for inducing differentiation into insulin-producing cells according to any one of claims 1 to 8 and a mixture of patient-derived blood as an active ingredient to a subject in need.

19. A method for preventing or treating diabetes mellitus, the method comprising administering a composition comprising insulin-producing cells differentiated by the method according to claim 12 as an active ingredient to a subject thereof.
